(19) United States
(12) Patent Application Publication
(10) Pub. No.: US 2008/0243121 A1
(43) Pub. Date: Oct. 2, 2008

(54) CURATIVE TREATMENT SYSTEM, CURATIVE TREATMENT DEVICE, AND TREATMENT METHOD FOR LIVING TISSUE USING ENERGY

(76) Inventors: Tomoyuki TAKASHINO, Hino-shi (JP); Toru NAGASE, Tachikawa-shi (JP); Koji IIDA, Sagamihara-shi (JP); Mai WAKAMATSU, Hino-shi (JP)

Correspondence Address: OSTROLENK FABER GERB & SOFFEN, 1180 AVENUE OF THE AMERICAS, NEW YORK, NY, 100368403, US

(21) Appl. No.: 11/695,133

(22) Filed: Apr. 2, 2007

(51) Int. Cl. A61B 18/14

(52) U.S. Cl. 606/52

(57) ABSTRACT

A treatment system that applies energy to a living tissue includes first and second holding members, an operating section, an energy source, and a plurality of energy applying portions that apply energy supplied from the energy source. Each of the first and second holding members has a holding surface to hold the living tissue. The operating section operates a relative movement of at least one of the first and second holding members with respect to the other. The energy source supplies energy to at least one of the first and second holding members. The plurality of energy applying portions are provided on the holding surface of at least one of the first and second holding members, and control density of energy applied to a living tissue held by the first and second holding members.

### BACKGROUND OF THE INVENTION

1. Field of the Invention

The present invention relates to a treatment system, a treatment device, and a treatment method for a living tissue using energy that enable energy to function with respect to a living tissue in a state where the living tissue is held.

2. Description of the Related Art

US Patent Application Publication No. 2005/0113828 A1 discloses electro-surgical instruments including a pair of juxtaposed jaw members each having an electroconductive surface. An over-shoe having a plurality of apertures is arranged in the pair of jaw members of the electro-surgical instruments. The over-shoe has, e.g., insulating properties. Therefore, energy for a treatment is supplied to a living tissue from the jaw members through the apertures of the over-shoe. Further, the apertures of the over-shoe are arranged in two rows along a longitudinal direction of the over-shoe.

### BRIEF SUMMARY OF THE INVENTION

According to a first aspect of the present invention, there is provide a treatment system that applies energy to a living tissue, the system includes:

first and second holding members each having a holding surface to hold the living tissue;

an operating section that operates a relative movement of at least one of the first and second holding members with respect to the other;

an energy source that supplies energy to at least one of the first and second holding members; and

a plurality of energy applying portions that apply energy supplied from the energy source, the plurality of energy applying portions being provided on the holding surface of at least one of the first and second holding members and controlling density of energy applied to the living tissue held by the first and second holding members.

According to a second aspect of the present invention, there is provided a treatment device that applies energy to a living tissue, the device includes:

a holding section that holds the living tissue, the holding section including:

first and second holding members that are relatively movable with respect to each other; and

a plurality of energy applying portions that are provided on at least one of the first and second holding members and connected with an energy source, the energy applying portions being provided on at least one of the first and second holding members and controlling density of energy applied to the living tissue when applying the energy to the living tissue held by the first and second holding members.

According to a third aspect of the present invention, there is provided a treatment method for a living tissue using an energy, the method includes:

holding the living tissue;

applying energy to the living tissue to denature the living tissue; and

increasing energy density at a desired position where the held living tissues denatures by the energy applied to the living tissue.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1A is a schematic view showing a treatment system according to a first embodiment of the present invention;

FIG. 1B is a schematic view when the treatment system according to the first embodiment is used to perform a bipolar type treatment;

FIG. 2A is a schematic longitudinal sectional view showing a shaft and a state where a first holding member and a second holding member of a holding section in an electro-surgical device according to the first embodiment are closed;

FIG. 2B is a schematic longitudinal sectional view showing the shaft and a state where the second holding member of the holding section are opened with respect to the first holding member in the electro-surgical device according to the first embodiment;

FIG. 3A is a schematic plan view showing the first holding member on a side close to the second holding member in the holding section of the electro-surgical device according to the first embodiment;

FIG. 3B is a schematic longitudinal sectional view showing the first holding member taken along a line 3B-3B depicted in FIG. 3A in the holding section of the electro-surgical device according to the first embodiment;

FIG. 3C is a schematic cross sectional view cut along the 3C-3C line of FIG. 3A, showing the first holding member in the holding section of the electro-surgical device according to the first embodiment;

FIG. 4A is a schematic view showing a surface of a main body of the first holding member in the holding section of the electro-surgical device according to the first embodiment and temperature distribution of a living tissue when energy is applied to the living tissue from electrodes on the surface of the main body of the first holding member;

FIG. 4B is a schematic view showing a prior art for comparison with the schematic view showing the surface of the main body of the first holding member in the holding section of the electro-surgical device according to the first embodiment and the temperature distribution of the living tissue when energy is applied to the living tissue from the electrodes on the surface of the main body of the first holding member depicted in FIG. 4A;

FIG. 5A is a schematic view when a treatment system according to the first embodiment is used to perform a bipolar type treatment;

FIG. 5B is a schematic view when the treatment system according to the first embodiment is used to perform a monopolar type treatment;

FIG. 5C is a schematic view when the treatment system according to the first embodiment is used to perform a monopolar type treatment;

FIG. 6 is a schematic view showing a modification of the treatment system according to the first embodiment of the present invention;

FIG. 7 is a schematic plan view showing a first holding member on a side close to a second holding member in a holding section of an electro-surgical device according to a second embodiment of the present invention;

FIG. 8 is a schematic plan view showing a first holding member on a side close to a second holding member in a holding section of an electro-surgical device according to a third embodiment of the present invention;

FIG. 9 is a schematic view showing a treatment system according to a fourth embodiment of the present invention;

FIG. 10A is a schematic longitudinal sectional view showing a shaft and a state where a first holding member and a second holding member of a holding section in an electro-surgical device according to the fourth embodiment are closed;

FIG. 10B is a schematic longitudinal sectional view showing the shaft and a state where the second holding member of the holding section are opened with respect to the first holding member in the electro-surgical device according to the fourth embodiment;

FIG. 11 is a schematic plan view showing the first holding member on a side close to the second holding member in the holding section of the electro-surgical device according to the fourth embodiment;

FIG. 12 is a schematic view showing a treatment system according to a fifth embodiment of the present invention;

FIG. 13A is a schematic longitudinal sectional view showing a state in which a main body side holding portion engages with a detachable side holding portion and the detachable side holding portion is disposed separated from the main body side holding portion of an electro-surgical device according to the fifth embodiment;

FIG. 13B is a schematic longitudinal sectional view showing a state in which the main body side holding portion engages with the detachable side holding portion and the detachable side holding portion is disposed close to the main body side holding portion of the electro-surgical device according to the fifth embodiment;

FIG. 13C is an enlarged schematic longitudinal sectional view showing a part of main body side holding portion denoted by reference character 13C in the electro-surgical device according to the fifth embodiment depicted in FIG. 13A;

FIG. 13D is an enlarged schematic longitudinal sectional view showing a part of the detachable side holding portion denoted by reference character 13D in the electro-surgical device according to the fifth embodiment depicted in FIG. 13A;

FIG. 14 is a schematic view showing the main body side holding portion in the electro-surgical device according to the fifth embodiment and temperature distribution of a living tissue when energy is supplied to the living tissue from electrodes on a surface of the main body side holding portion;

FIG. 15A is a schematic view showing the surface of the main body side holding portion in the holding section of the electro-surgical device according to the fifth embodiment and temperature distribution of a living tissue when energy is applied to the living tissue from electrodes on the surface of the main body side holding portion;

FIG. 15B is a schematic view showing a prior art for comparison with the schematic view showing the surface of the main body side holding portion in the holding section of the electro-surgical device according to the fifth embodiment and the temperature distribution of the living tissue when energy is applied to the living tissue from the electrodes on the surface of the main body side holding portion depicted in FIG. 15A;

FIG. 16 is a schematic view showing a main body side holding portion in an electro-surgical device according to a sixth embodiment and temperature distribution of a living tissue when energy is applied to the living tissue from electrodes on a surface of the main body side holding portion;

FIG. 17 is a schematic view showing a main body side holding portion in an electro-surgical device according to a seventh embodiment and temperature distribution of a living tissue when energy is applied to the living tissue from electrodes on a surface of the main body side holding portion;

FIG. 18 is a schematic view showing a main body side holding portion in an electro-surgical device according to an eighth embodiment and temperature distribution of a living tissue when energy is applied to the living tissue from electrodes on a surface of the main body side holding portion;

FIG. 19 is a schematic view showing a main body side holding portion in an electro-surgical device according to a ninth embodiment and temperature distribution of a living tissue when energy is applied to the living tissue from electrodes on a surface of the main body side holding portion;

FIG. 20 is a schematic view showing a main body side holding portion in an electro-surgical device according to a tenth embodiment and temperature distribution of a living tissue when energy is applied to the living tissue from electrodes on a surface of the main body side holding portion; and

FIG. 21 is a schematic view showing a main body side holding portion in an electro-surgical device according to an eleventh embodiment and temperature distribution of a living tissue when energy is applied to the living tissue from electrodes on a surface of the main body side holding portion.

### DETAILED DESCRIPTION OF THE INVENTION

The best mode for carrying out the present invention will now be explained with reference to the accompanying drawings.

### First Embodiment

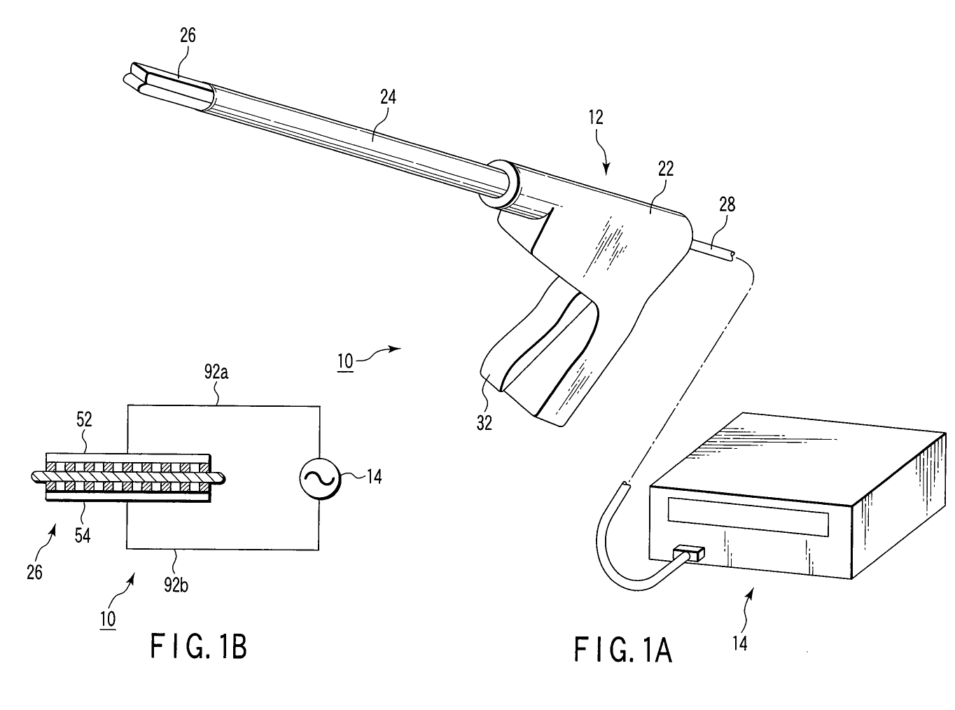

A first embodiment will now be explained with reference to FIGS. 1A to 6.

Here, as an example of an energy treatment device, a linear type bipolar electro-surgical device 12 which performs a treatment through, for example, an abdominal wall will be described.

As shown in FIGS. 1A and 1B, a treatment system 10 includes the electro-surgical device (a treatment device for curing) 12 and an energy source 14.

The electro-surgical device 12 includes a handle 22, a shaft 24 and an openable/closable holding section 26. The handle 22 is connected with the energy source 14 via a cable 28. The energy source 14 is connected to a foot switch and a handle switch (not shown). Therefore, these foot and hand switches are operated by an operator to switch ON/OFF of the supply of energy from the energy source 14 to the electro-surgical device 12.

The handle 22 is substantially formed into an L-shape. The shaft 24 is disposed on one end of the handle 22. The cable 28 is extended from a proximal side of the handle 22 disposed coaxially with the shaft 24.

On the other hand, the other end of the handle 22 is a grip held by the operator. The handle 22 includes a holding section opening/closing knob 32 arranged on the other end of the handle 22. The holding section opening/closing knob 32 is connected with a proximal end of a sheath 44 described later of the shaft 24 substantially at the center of the handle 22. When the holding section opening/closing knob 32 is allowed to come close to or come away from the other end of the handle 22, the sheath 44 moves along an axial direction of the shaft 24.

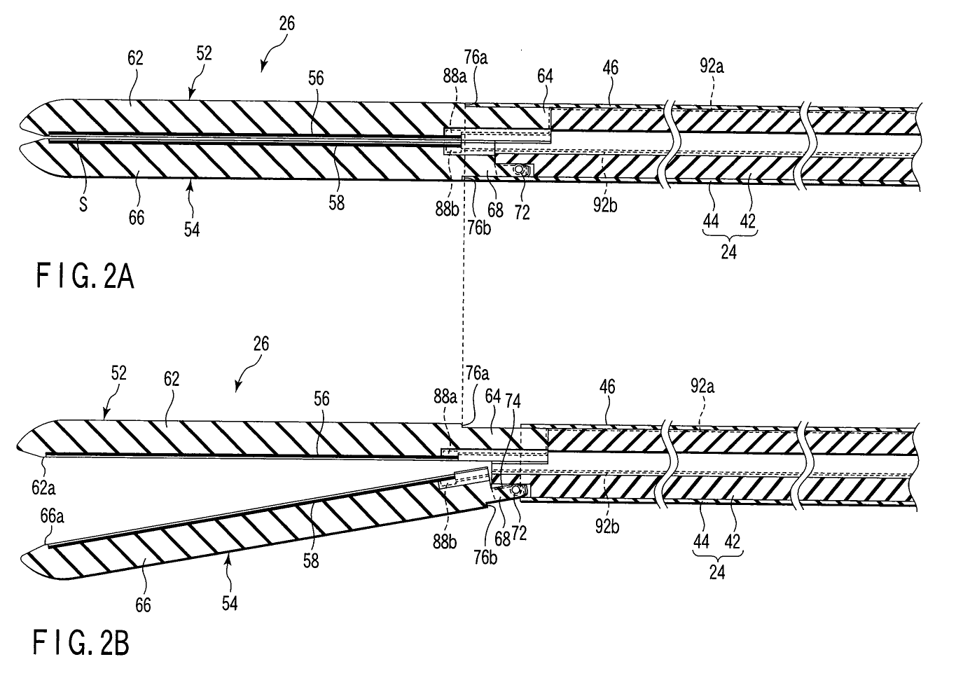

As shown in FIGS. 2A and 2B, the shaft 24 includes a cylindrical member 42 and the sheath 44 slidably disposed outside the cylindrical member 42. A proximal end of the cylindrical member 42 is fixed to the handle 22. The sheath 44 is slidable along an axial direction of the cylindrical member 42.

Outside the cylindrical member 42, concave portion 46 is formed along the axial direction of the cylindrical member 42. The concave portion 46 is provided with a first conducting line 92a connected to a first high-frequency electrode 56 described later. A second conducting line 92b connected to a second high-frequency electrode 58 described later is passed through the cylindrical member 42.

It is to be noted that the first high-frequency electrode plate 56 is electrically connected with a first electrode connector 88a. This first electrode connector 88a is connected with the cable 28 extended from the handle 22 via a first energization line 92a. The second high-frequency electrode plate 58 is electrically connected with a second electrode connector 88b. This second electrode connector 88a is connected with the cable 28 extended from the handle 22 via a second energization line 92b.

As shown in FIGS. 1A, 2A, and FIG. 2B, the holding section 26 is disposed at a distal end of the shaft 24. As shown in FIGS. 2A and 2B, the holding section 26 includes a first holding portion 52, a second holding portion 54, the first high-frequency electrode 56 as an output portion or an energy applying portion, and the second high-frequency electrode 58 as another output portion or another energy release portion.

It is preferable that the first holding portion 52 and the second holding portion 54 entirely have insulating properties, respectively. The first holding portion 52 integrally includes a first holding portion main body (hereinafter referred to mainly as the main body) 62 provided with the first high-frequency electrode 56 and a base portion 64 disposed at a proximal end of the main body 62. The second holding portion 54 integrally includes a second holding portion main body 66 provided with the second high-frequency electrode 58 and a base portion 68 disposed at a proximal end of the main body 66.

The base portion 64 of the first holding portion 52 is fixed to a distal end of the cylindrical member 42 of the shaft 24. On the other hand, the base portion 68 of the second holding portion 54 is rotatably supported at the distal end of the cylindrical member 42 of the shaft 24 by a support pin 72 disposed in a direction crossing the axial direction of the shaft 24 at right angles. The second holding portion 54 can rotate around an axis of the support pin 72 to open or close with respect to the first holding portion 52. Moreover, the second holding portion 54 is urged so as to open with respect to the first holding portion 52 by an elastic member 74 such as a leaf spring.

Outer surfaces of the main bodies 62 and 66 of the first holding portion 52 and the second holding portion 54 are formed into smooth curved surfaces. Similarly, outer surfaces of the base portions 64 and 68 of the first holding portion 52 and the second holding portion 54 are also formed into smooth curved surfaces. While the second holding portion 54 is closed with respect to the first holding portion 52, sections of the main bodies 62, 66 of the support members 52, 54 are formed into substantially circular or elliptic shapes. When the second holding portion 54 is closed with respect to the first holding portion 52, the base portions 64, 68 are formed into cylindrical shapes. In this state, a diameter of each of the proximal ends of the main bodies 62, 66 of the first holding portion 52 and the second holding portion 54 is formed to be larger than a diameter of each of the base portions 64, 68. Moreover, stepped portions 76a, 76b are formed between the main bodies 62, 66 and the base portions 64, 68, respectively.

Here, in the first holding portion 52 and the second holding portion 54, while the second holding portion 54 is closed with respect to the first holding portion 52, a substantially circular or elliptic outer peripheral surface formed by combining the base portions 64, 68 of the holding portions 52, 54 is substantially the same plane as that of an outer peripheral surface of the distal end of the cylindrical member 42, or a diameter of the outer peripheral surface is formed to be slightly larger than that of the outer peripheral surface of the distal end of the cylindrical member 42. Therefore, the sheath 44 can be slid with respect to the cylindrical member 42 to cover the base portions 64, 68 of the first holding portion 52 and the second holding portion 54 with a distal end of the sheath 44. In this state, as shown in FIG. 2A, the first holding portion 52 and the second holding portion 54 close against an urging force of the elastic member 74. On the other hand, the sheath 44 is slid toward the proximal end of the cylindrical member 42 from the state in which the base portions 64, 68 of the first holding portion 52 and the second holding portion 54 are covered with the distal end of the sheath 44. In this case, as shown in FIG. 2B, the second holding portion 54 is opened with respect to the first holding portion 52 by the urging force of the elastic member 74.

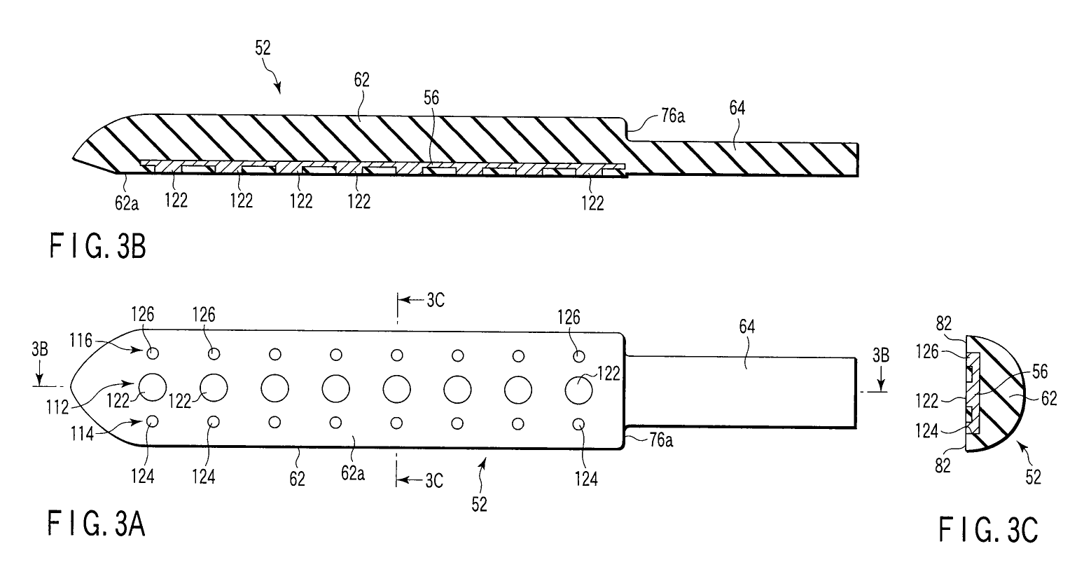

As shown in FIGS. 3B and 3C, the first high-frequency electrode plate 56 is arranged in the main body 62 of the first holding member 52. As shown in FIG. 3A, the first high-frequency electrode plate 56 includes a first high-frequency electrode group (which will be referred to as a first electrode group hereinafter) 112, a second high-frequency electrode group (which will be referred to as a second electrode group hereinafter) 114, and a third high-frequency electrode group (which will be referred to as a third electrode group hereinafter) 116 in each of columns. As shown in FIG. 3B, the first electrode group 112, the second electrode group 114, and the third electrode group 116 include a plurality of (eight in each group in this example) electrodes 122, 124, and 126 each having a convex cross section along a longitudinal direction of the main body 62 like spots.

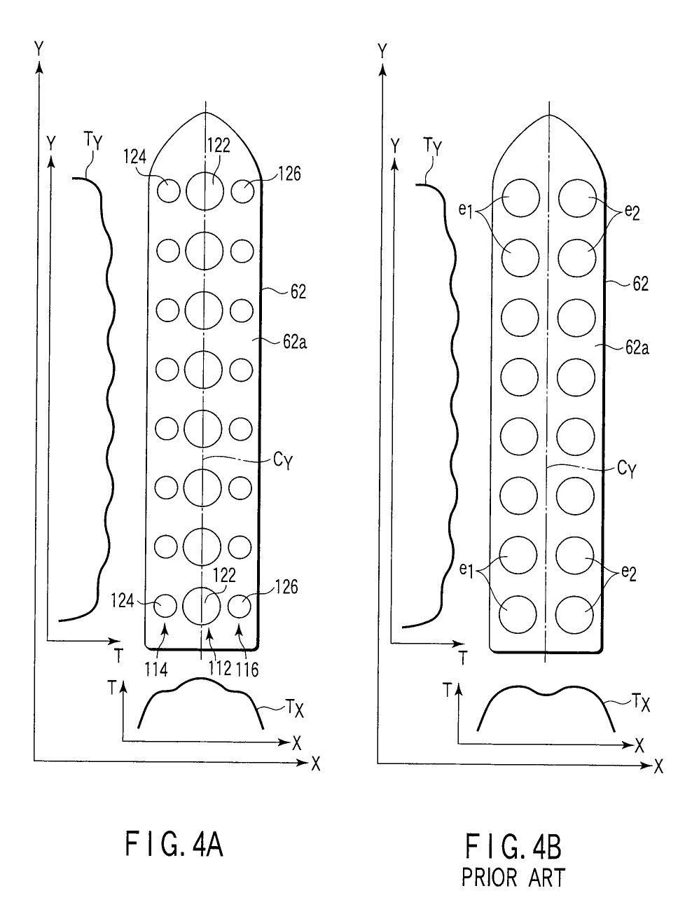

The first electrode group 112 is arranged in a region (a first region) along a central axis C_{Y} of the main body 62 in the longitudinal direction (a Y axis direction in FIG. 4A). The second electrode group 114 is arranged in a region (a second region) away from the central axis C_{Y} of the main body 62 by a predetermined distance. Likewise, the third electrode group 116 is arranged in a region (the second or third region) away from the central axis C_{Y} of the main body 62 by a predetermined distance. That is, the first electrode group 112, the second electrode group 114, and the third electrode group 116 are respectively arranged in the Y axis direction in FIG. 4A.

It is to be noted that the second electrode group 114 and the third electrode group 116 are arranged at substantially symmetrical positions with respect to the central axis C_{Y} of the main body 62. That is, the second electrode group 114 and the third electrode group 116 are arranged at substantially symmetrical positions with respect to the first electrode group 112. In other words, a distance between the first electrode group 112 and the second electrode group 114 is substantially equal to a distance between the first electrode group 112 and the third electrode group 116. Further, one electrode 122 of the first electrode group 112, one electrode 124 of the second electrode group 114, and one electrode 126 of the third electrode group 116 are arranged on the same axis in the X axis direction in FIG. 4A (see FIG. 3C).

Exposed areas of the respective electrodes 124 and 126 in the second electrode group 114 and the third electrode group 116 are substantially equal to each other. An exposed area of each electrode 122 in the first electrode group 112 is larger than the exposed area of each of the electrodes 124 and 126 in the second electrode group 114 and the third electrode group 116. Further, a distance between the respective electrodes 122 in the first electrode group 112, a distance between the respective electrodes 124 in the second electrode group 114, and a distance between the respective electrodes 126 in the third electrode group 116 are substantially equal to each other.

Here, it is assumed that outputs from the respective electrodes 122, 124, and 126 in the first to third electrode groups 112, 114, and 116 per unit area are in proportion to each other.

Furthermore, the second high-frequency electrode plate 58 is also arranged on the second holding member 54 to be symmetrical to the first holding member 52. A detailed explanation of this structure will be omitted.

A function of a treatment system 10 according to this embodiment will now be explained.

As shown in FIG. 2A, in a state where the second holding member 54 is closed with respect to the first holding member 52, the holding section 26 and the shaft 24 of the electro-surgical device 12 are inserted into, e.g., an abdominal cavity through an abdominal wall. The holding section 26 of the electro-surgical device 12 is opposed to a living tissue as a treatment target.

The holding section opening/closing knob 32 of the handle 22 is operated to hold the living tissue as a treatment target by using the first holding member 52 and the second holding member 54. At this time, the sheath 44 is moved toward a proximal side of the shaft 24 on the cylindrical member 42. A space between the base portions 64 and 68 cannot be maintained in a cylindrical shape due to an urging force of the elastic member 74, and the second holding member 54 is then opened with respect to the first holding member 52.

Moreover, the living tissue as a treatment target is arranged between the first high-frequency electrode plate 56 of the first holding member 52 and the second high-frequency electrode plate 58 of the second holding member 54. In this state, the holding section opening/closing knob 32 of the handle 22 is operated. At this time, the sheath 44 is moved to a distal end side of the shaft 24 with respect to the cylindrical body 42. The base portions 64 and 68 are closed to form the cylindrical shape therebetween against the urging force of the elastic member 74 by using the sheath 44. Therefore, the first holding member main body 62 integrally formed on the base portion 64 and the second holding member main body 66 integrally formed on the base portion 68 are closed. That is, the second holding member 54 is closed with respect to the first holding member 52. Therefore, the living tissue as a treatment target is grasped between the first holding member 52 and the second holding member 54.

At this time, the living tissue as a treatment target is in contact with both the electrodes 122, 124, and 126 of the first high-frequency electrode plate 56 provided on the first holding member 52 and the electrodes 122, 124, and 126 of the second high-frequency electrode plate 58 provided on the second holding member 54. A surrounding tissue of the living tissue as a treatment target is appressed against both a contact surface of the edge portion 82 of the first holding member and a contact surface of the edge portion (not shown) of the second holding member 54.

In this state, the foot switch or the hand switch is operated. Energy is respectively applied to the first high-frequency electrode plate 56 and the second high-frequency electrode plate 58 from the energy source 14 through the cable 28, the first and second energization lines 92a and 92b, and the first and second energization connectors 88a and 88b.

Since the treatment system 10 according to the embodiment is of a bipolar type as shown in FIGS. 1A and 1B, the electrodes 122, 124, and 126 of the first high-frequency electrode plate 56 apply a high-frequency current to a space between themselves and the electrodes 122, 124, and 126 of the second high-frequency electrode plate 58 via the living tissue as a treatment target. Therefore, the living tissue held between the main body 62 of the first holding member 52 and the main body 66 of the second holding member 54 is heated.

As this time, as shown in FIGS. 3A and 4A, each electrode 122 in the first electrode group 112 has a larger contact area with respect to the living tissue than that of each electrode 124 or each electrode 126 in the second electrode group 114 or the third electrode group 116. Therefore, energy supplied to the living tissue from each electrode 122 in the first electrode 112 is larger than energy applied to the living tissue from each electrode 124 and each electrode 126 in the second electrode group 114 and the third electrode 116.

Further, the living tissue that is in contact with the second electrode group 114 or the third electrode group 116 is away from the central axis C_{Y} and close to the outside of the holding section 26. Therefore, the living tissue is affected by the outside of the holding section 26 having temperature far lower than that of the living tissue present between the first holding member 52 and the second holding member 54. That is, heat of the living tissue grasped by the holding section 26 is taken by a peripheral environment at a position close to the edge portion of the holding section 26.

Therefore, density of the energy supplied to the living tissue from the holding section 26 is high at a position near the central axis C_{Y}, and becomes lower than that at a position near the central axis C_{Y} as distanced from the central axis C_{Y}. Therefore, energy distribution of the first electrode group 112 is higher than those of the second and third electrode groups 114 and 116. That is, temperature distribution (the energy density) T_{X} of the living tissue held by the holding section 26 in the X axis direction is high at a position near the central axis C_{Y}, and becomes low as distanced from the central axis C_{Y}. Therefore, a temperature gradient of the living tissue in the holding section 26 along the X axis direction is large.

In other words, in the X axis direction of the holding section 26, the living tissue receives large energy at a position near the central axis C_{Y}, and receives smaller energy than that at the position near central axis C_{Y} as distanced from the central axis C_{Y}. Therefore, for example, when welding the living tissue, a treatment of, e.g., denaturing and conjugating the living tissue can be assuredly performed near the central axis C_{Y} of the main body 62 of the first holding member 52. Contrary, denaturation of a surrounding tissue can be avoided as much as possible.

Meanwhile, electrodes e₁ and e₂ are arranged in two columns at positions away from a central axis C_{Y} by an equal distance on a main body 62 of a first holding member 52 according to the prior art depicted in FIG. 4B. When giving a treatment to a living tissue grasped by such a first holding member 52, temperature distribution T_{X} shown in FIG. 4B is demonstrated, for example. The temperature distribution T_{X} has a depression at a central part (near the central axis C_{Y}), and temperature of the living tissue at a position corresponding to an edge portion of the main body 62 of the first holding member 52 is also reduced due to an influence of the outside of the holding section 26 as compared the central part with the position corresponding to the edge portion of the main body 62 of the first holding member 52.

As explained above, according to this embodiment, the following effect can be obtained.

As shown in FIG. 4A, the first electrode group 112 is arranged on the central axis C_{Y} of the main body 62 of the first holding member 52, and the second electrode group 114 and the third electrode group 116 are arranged at positions away from the central axis C_{Y}. Further, a contact area of each electrode 122 in the first electrode group 112 with respect to the living tissue is set to be larger than those of the respective electrodes 124 and 126 in the second electrode group 114 and the third electrode group 116. That is, an amount of energy applied to the living tissue from each electrode 122 in the first electrode group 112 is larger than an amount of energy applied to the living tissue from each electrode 124 or 126 in the second or third electrode group 114 or 115.

Then, a larger temperature gradient can be obtained, for example, in the temperature distribution T_{X} in the X axis direction demonstrated by the living tissue due to the main body 62 of the first holding member 52 depicted in FIG. 4A, a position corresponding to the central part (near the central axis C_{Y}) of the main body 62 of the first holding member 52 can be raised and the position corresponding to the edge part of the same can be set lower than the central side as compared with the temperature distribution T_{X} of the prior art shown in FIG. 4B. That is, the temperature gradient of the temperature distribution T_{X} in the X axis direction given to the living tissue by the main body 62 of the first holding member 52 depicted in FIG. 4A can be set larger than a temperature gradient of the temperature distribution T_{X} of the prior art shown in FIG. 4B. In particular, the temperature gradient at the central part can be increased. Then, an arrangement of each electrode 122 in the X axis direction of the main body 62 of the first holding member 52 can assuredly enable denaturation and fusion of the living tissue, and an arrangement of each electrode 124 and each electrode 126 can avoid an influence given to a surrounding tissue as much as possible.

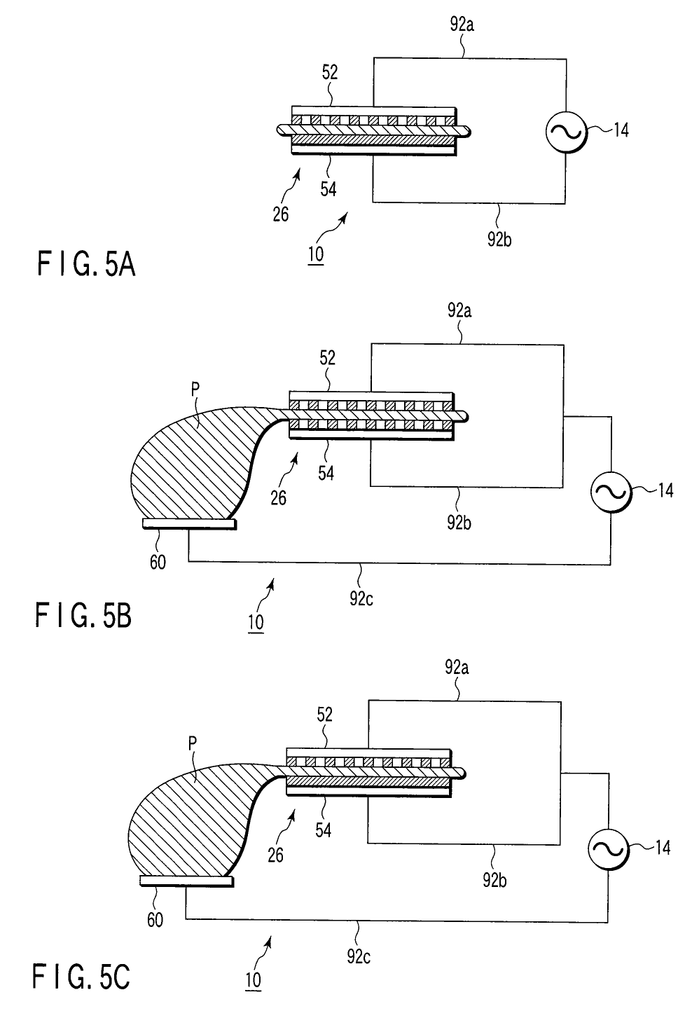

It is to be noted that the holding section 26 when the structure of the main body 62 of the first holding member 52 and the structure of the main body 66 of the second holding member 54 are symmetrical (the same) has been explained in this embodiment. Besides, as shown in FIG. 5A, it is also preferable to adopt the above-explained configuration for the main body 62 of the first holding member 52 and use a second high-frequency electrode 58 like one plane that is entirely exposed on a holding surface 66a on a side close to the first holding member 52. Even in this case, since the structure of the first high-frequency electrode plate 56 provided on the main body 62 of the first holding member 52 is the same, a treatment can be performed to obtain the same temperature distribution T_{X} when carrying out the treatment with respect to the living tissue.

Although using the bipolar type electro-surgical device 12 has been explained in this embodiment, using a monopolar type electro-surgical device is also preferable as shown in FIGS. 5B and 5C. In this case, a counter electrode plate 60 is attached to a patient P who is a treatment target. This counter electrode plate 60 is connected with the energy source 14 via the energization line 92c. Further, the first high-frequency electrode plate 56 arranged on the main body 62 of the first holding member 52 and the second high-frequency electrode plate 58 arranged on the main body 66 of the second holding member 54 are in the same potential state where the first and the second energization lines 92a and 92b are electrically connected with each other. In this case, an area of the living tissue that is in contact with the first and the second high-frequency electrode plates 56 and 58 is small. Therefore, a current density is high, but a current density of the counter electrode plate 60 is low. Therefore, the living tissue held by the holding section 26 generates heat, but heat generation of the living tissue that is in contact with the counter electrode plate 60 is vanishingly small. Therefore, the part grasped by the holding section 26 alone is heated and, at this time, the living tissue held by the holding section 26 can obtain the temperature distribution T_{X} with a large temperature gradient as explained above. That is, the temperature distribution at the central part is higher than the temperature distribution at the periphery along the X axis direction of the main bodies 62 and 66 of the first and second holding members 52 and 54.

Furthermore, although not shown, when the monopolar type electro-surgical device is used, arranging the high-frequency electrodes on one of the first holding member 52 and the second holding member 54 alone is also preferable.

Although using the high-frequency electrodes has been explained in this embodiment, ultrasonic transducers or heater elements (not shown) can be used as energy applying portions in place of adopting the high-frequency electrodes. When using the ultrasonic transducers or the heater elements in this manner, arranging the ultrasonic transducers or the heater elements on at least one of the first and second holding members 52 and 54 enables performing a treatment.

When using, e.g., spot-like ultrasonic transducers in place of the high-frequency electrodes, subjecting these ultrasonic transducers to ultrasonic vibration enables performing a treatment with respect to the living tissue that is in contact with a surface of each ultrasonic transducer like an example where the high-frequency electrodes are used to effect a treatment.

Moreover, when using, e.g., spot-like heater elements in place of the high-frequency electrodes, allowing heat generation from these heater elements enables performing a treatment with respect to the living tissue that is in contact with a surface of each heater element like an example where the high-frequency electrodes are used to effect a treatment.

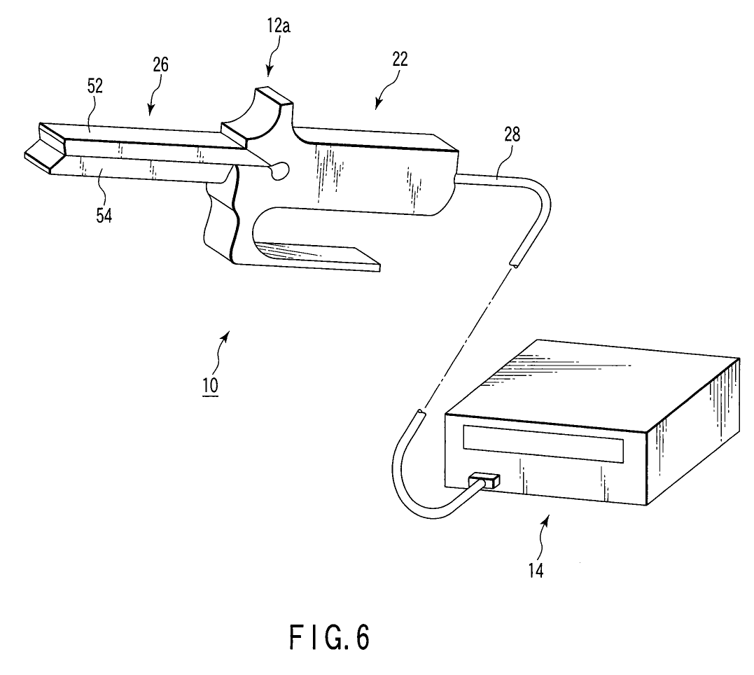

In this embodiment, the linear electro-surgical device 12 for treating the living tissue of the abdominal cavity (in a body) through the abdominal wall has been described as an example. However, for example, as shown in FIG. 6, an open type linear electro-surgical device (a treatment device for curing) 12a may be used which extracts a treatment target tissue out of the body through the abdominal wall to treat the tissue.

The electro-surgical device 12a includes a handle 22 and a holding section 26. That is, unlike the electro-surgical device 12 for treating the tissue through the abdominal wall, the shaft 24 (see FIG. 1A) is omitted. On the other hand, a member having a function similar to that of the shaft 24 is disposed in the handle 22. Therefore, the device can be used in the same manner as in the electro-surgical device 12 described above with reference to FIG. 1A.

### Second Embodiment

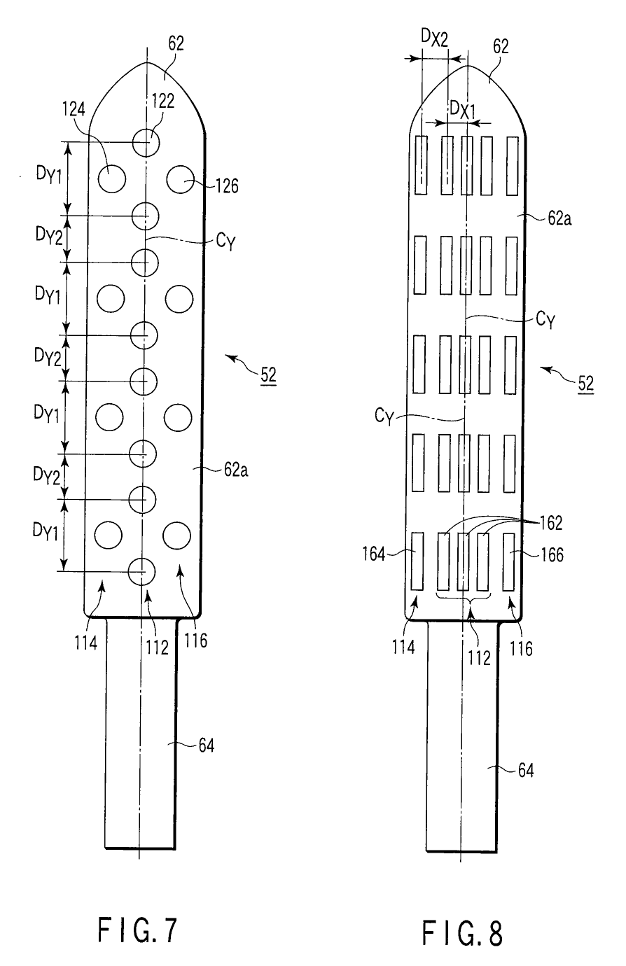

A second embodiment will now be explained with reference to FIG. 7. This embodiment is a modification of the first embodiment, and like reference numerals denote members equal to those in the members explained in the first embodiment, thereby omitting a detailed explanation thereof.

As shown in FIG. 7, respective electrodes 122, 124, and 126 in first to third electrode groups 112, 114, and 116 have the same area.

Two types of distances D_{Y1} and D_{Y2} are provided between centers of the respective electrodes 122 in the first electrode group 112. The distance D_{Y1} is a distance between a center of the electrode 122 at the outermost end in a Y axis direction and a center of the next inner electrode 122 adjacent to this end. Additionally, the distance D_{Y2} is a distance between the center of the next inner electrode 122 adjacent to the outermost end in the Y axis direction and a center of the next inner electrode 122 adjacent to the former electrode.

On the other hand, the second electrode group 114 includes four electrodes 124. Distances between centers of the electrodes 124 adjacent to each other are equal. Further, each electrode 124 in the second electrode group 114 is arranged at a position that is between the centers of the electrodes 122 arranged with a gap having the distance D_{Y1} therebetween and is away from a central axis C_{Y} by a predetermined distance. It is to be noted that, in regard to an arrangement of each electrode 126 in the third electrode group 116, distances between centers of the electrodes 126 adjacent to each other are equal like the second electrode group 114. Furthermore, the second and third electrode groups 114 and 116 are provided at symmetrical positions with the central axis C_{Y} at the center.

Therefore, a holding surface 62a of a main body 62 of a first holding member 52 has high density since the number of the electrodes 122 in the first electrode group 112 near the central axis C_{Y} in an X axis direction is large, and densities of the second and third electrode groups 114 and 116 away from the central axis C_{Y} are low.

Therefore, energy distribution of the first electrode group 112 is higher than those of the second and third electrode groups 114 and 116. That is, temperature distribution (energy density) T_{X} of a living tissue held by the holding section 26 in the X axis direction is high near the central axis C_{Y}, and becomes lower as away from the central axis C_{Y}. Therefore, a temperature gradient of the living tissue along the X axis direction in the holding section 26 is large.

In other words, the living tissues receives large energy at a position near the central axis C_{Y} in the X axis direction of the holding section 26, and receives smaller energy than that at the position near the central axis C_{Y} as away from the central axis C_{Y}. Therefore, when, e.g., welding the living tissue, a treatment of, e.g., denaturing and conjugating the living tissue can be assuredly performed near the central axis C_{Y} of the main body 62 of the first holding member 52. Contrary, denaturation of a surrounding tissue can be avoided as much as possible.

### Third Embodiment

A third embodiment will now be explained with reference to FIG. 8. This embodiment is a modification of the first and the second embodiments, and like reference numerals denote members equal to those explained in the first and the second embodiments, thereby omitting a detailed explanation.

As shown in FIG. 8, a first electrode group 112 includes a total of 15 rectangular electrodes 162 in five rows and three columns in this embodiment. A longitudinal direction of each electrode 162 is a Y axis direction. The respective electrodes 162 are arranged in an X axis direction and a Y axis direction at equal intervals.

Each of second and third electrode groups 114 and 116 includes five rectangular electrodes 164 or 166. The electrodes 164 or 166 in the second or third electrode group 114 or 116 are provided in a single row along the X axis direction. The respective electrodes 164 and the respective electrodes 166 are arranged at equal intervals. A longitudinal direction of the respective electrodes 164 in the second electrode group 114 or the respective electrodes 166 in the third electrode group 116 is the Y axis direction.

It is to be noted that the respective electrodes 162 in the first electrode group 112 and the respective electrodes 164 and 166 in the second and third electrode groups 114 and 116 have substantially the same areas.

Further, a distance D_{X1} between the electrode 162 in the first electrode group 112 arranged on the central axis C_{Y} in the X axis direction of a main body 62 of a first holding member 52 (the electrode 162 in a second column) and the electrode 162 in a first column close the second electrode group 114 or the electrode 162 in a third column close to the third electrode group 116 is shorter than a distance D_{X2} between the electrode 162 in the first column in the first electrode group 112 and the electrode 164 in the second electrode group 114. This is also applied to a relationship between the first electrode group 112 and the third electrode group 116.

That is, the example where the number of the electrodes 122 in the first electrode group 112 along the Y axis direction is larger than the number of the electrodes 124 or 126 in the second or the third electrode group 114 or 116 has been explained in the second embodiment, but this embodiment provides an example where the number of electrodes 162 in the first electrode group 112 in the X axis direction is larger than the number of the electrodes 164 or 166 in the second or the third electrode group 114 and 116.

A function of a treatment system 10 according to this embodiment will now be explained.

As shown in FIG. 8, although the number of the columns of the respective electrodes 162 in the first electrode group 112 is three, the number of the column of the respective electrodes 164 or 166 in the second electrode group 114 or the third electrode group 116 is one. Therefore, a holding surface 62a of a main body 62 of a first holding member 52 has high density since the number of the electrodes 122 in the first electrode group 112 near the central axis C_{Y} in the X axis direction is large, and each of the second and third electrode groups 114 and 116 away from the central axis C_{Y} has low density.

Therefore, energy distribution of the first electrode group 112 is higher than those of the second and third electrode groups 114 and 116. That is, temperature distribution (energy density) T_{X} in the X axis direction of a living tissue grasped by the holding section 26 is high near the central axis C_{Y}, and becomes low as away from the central axis C_{Y}. Therefore, a temperature gradient in the X axis direction of the living tissue in the holding section 26 is large.

In other words, the living tissue receives large energy at a position near the central axis C_{Y} along the X axis direction of the holding section 26, and receives smaller energy than that at the position near the central axis C_{Y} as away from the central axis C_{Y}. Therefore, when, e.g., welding the living tissue, a treatment of, e.g., denaturing and conjugating the living tissue can be assuredly performed near the central axis C_{Y} of the main body 62 of the first holding member 52, and denaturation of a surrounding tissue can be avoided as much as possible.

It is to be noted that the example where each of the electrodes 162, 164, and 166 in the first to third electrode groups 112, 114, and 116 has the rectangular shape has been explained in this embodiment, various shapes, e.g., an elliptic shape can be allowed.

Although the example where the electrodes 162 in the first electrode group 112 are provided in the three columns has been explained in this embodiment, it is also preferable for the first electrode group 112 to have a structure where the three electrodes 162 adjacent to each other along the X axis direction are formed as one electrode.

### Fourth Embodiment

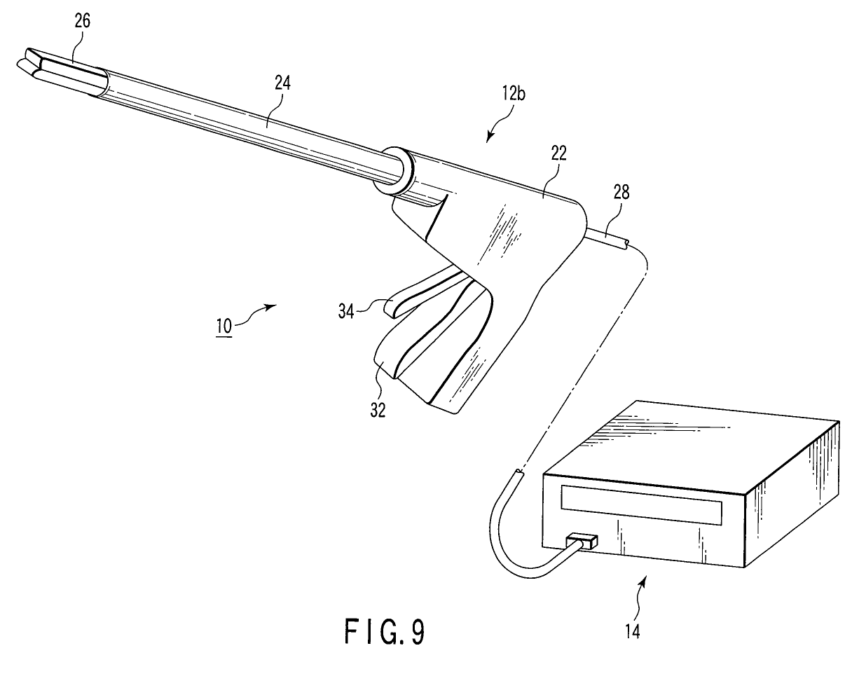

A fourth embodiment will now be explained with reference to FIGS. 9 to 11. This embodiment is a modification of the first to third embodiments, and like reference numerals denote members equal to those in the first to third embodiments or members having the same functions, thereby omitting a detailed explanation thereof.

As shown in FIG. 9, a handle 22 of an electro-surgical device (a treatment device for curing) 12b according to this embodiment is provided with a cutter driving knob 34 disposed along a holding section opening/closing knob 32.

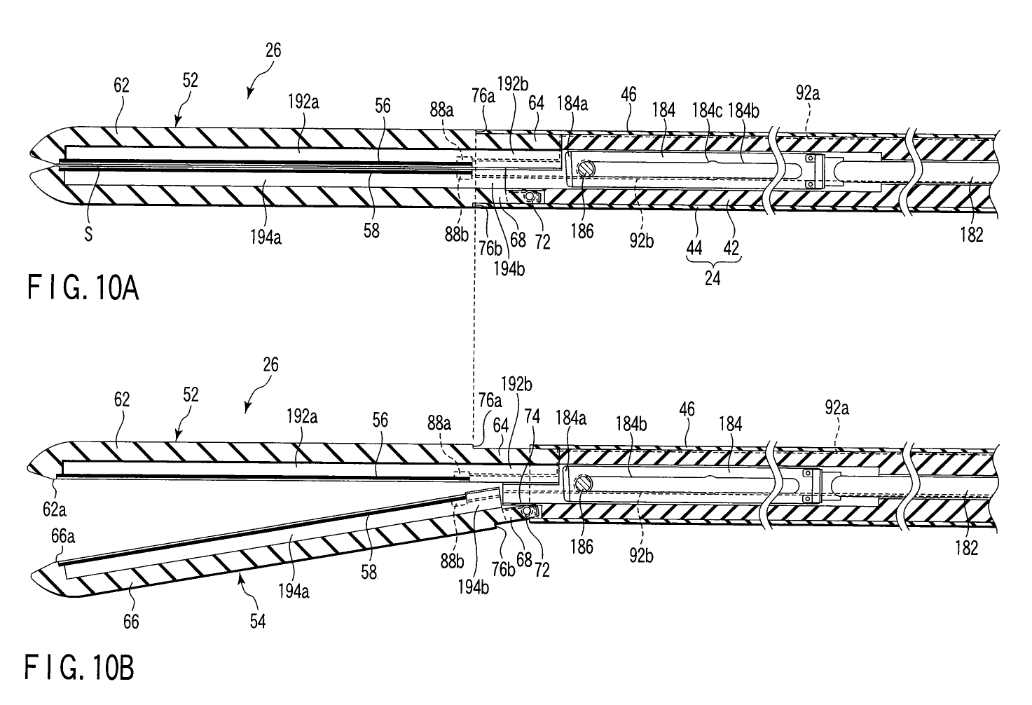

As shown in FIGS. 10A and 10B, a driving rod 182 is movably disposed along an axial direction of a cylindrical member 42 in the cylindrical member of a shaft 24. A distal end of the driving rod 182 is provided with a thin-plate-like cutter 184. Therefore, when the cutter driving knob 34 is operated, the cutter (an auxiliary curative device) 184 moves via the driving rod 182.

As shown in FIGS. 10A and 10B, a distal end of the cutter 184 is provided with a blade 184a, and the distal end of the driving rod 182 is fixed to a proximal end of the cutter 184. A longitudinal groove 184b is formed between the distal end and the proximal end of the cutter 184. Engagement portions 184c which engage with a movement regulation pin 186 are formed on one end of the longitudinal groove 184b, the other end and between one end and the other end. In the longitudinal groove 184b, the movement regulation pin 186 extending in a direction crossing the axial direction of the shaft 24 at right angles is fixed to the cylindrical member 42 of the shaft 24. Therefore, the longitudinal groove 184b of the cutter 184 moves along the movement regulation pin 186. In this case, the cutter 184 linearly moves. At this time, the cutter 184 is disposed along cutter guide grooves 192a, 192b, 194a and 194b of a first holding member 52 and a second holding member 54.

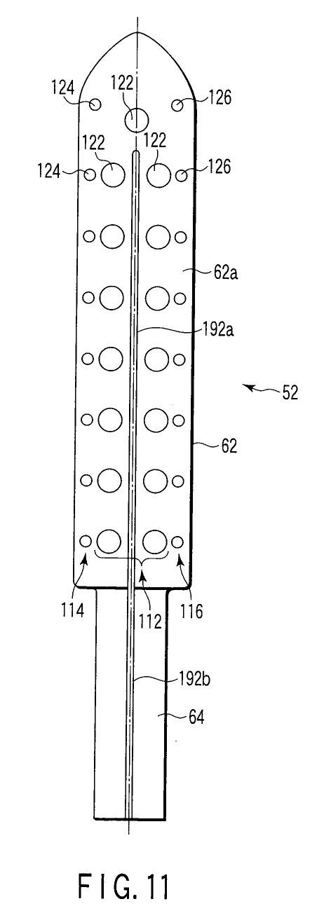

As shown in FIG. 11, cutter guide grooves 192a and 192b are formed on a central axis C_{Y} of a first holding member 52 on a side close to the second holding member 54. A distal end (an upper end) of the cutter guide groove 192a of a main body 62 of the first holding member 52 in FIG. 11 is present between, e.g., a distal end (an upper end) and a proximal end (a lower end) of the main body 62.

An electrode 122 at the uppermost end in a first electrode group 112 is arranged on the distal end side apart from the upper end of the cutter guide groove 192a. The remaining electrodes 122 in the first electrode groove 122 are symmetrically arranged with a central axis of the main body 62 having the cutter guide groove 192a provided therein at the center along a Y axis direction at equal intervals. Therefore, the remaining electrodes 122 in the first electrode group 112 are arranged to face the cutter guide groove 192a formed in the main body 62. In particular, an area of each electrode 122 in the first electrode group 112 is larger than those of respective electrodes 124 and 126 in second and third electrode groups 114 and 116.

A function of a treatment system 10 according to this embodiment will now be explained.

As shown in FIG. 11, each electrode 122 in the first electrode group 112 has a larger contract area for a living tissue than those of the respective electrodes 124 and 126 in the second electrode group 114 and the third electrode group 116. Therefore, energy applied to the living tissue from each electrode 122 in the first electrode group 112 is larger than energies applied to the living tissue from the respective electrodes 124 and 126 in the second electrode group 114 and the third electrode group 116.

Therefore, energy density applied to the living tissue by the holding section 26 is high near the central axis C_{Y} and becomes lower than that near the central axis C_{Y} as away from the central axis C_{Y}. Therefore, energy distribution of the first electrode group 112 is higher than those of the second and third electrode groups 114 and 116. That is, temperature distribution (energy density) T_{X} in the X axis direction of the living tissue held by the holding section 26 is high near the central axis C_{Y}, and is reduced as away from the central axis C_{Y}. Therefore, a temperature gradient in the X axis direction of the living tissue in the holding section 26 is large.

In other words, the living tissue receives large energy at a position near the central axis C_{Y} along the X axis direction of the holding section 26, and receives smaller energy than that at the position near the central axis C_{Y} as away from the central axis C_{Y}. Therefore, when, e.g., welding the living tissue, a treatment of, e.g., denaturing and conjugating the living tissue can be assuredly performed near the central axis C_{Y} of the main body 62 of the first holding member 52. Contrary, denaturation of a surrounding tissue can be avoided as much as possible.

Furthermore, after the living tissue is subjected to a heat treatment, the cutter driving knob 34 of the handle 22 is operated. Then, a cutter 174 moves toward the distal ends of the first holding member 52 and the second holding member 54. Since the cutter 174 has a blade 174a at a distal end thereof, thereby cutting the treated living tissue.

It is to be noted that the electrode 122 provided at the uppermost end in the first electrode group 112 in FIG. 11 is arranged on a lower side than the electrodes 124 and 126 provided at the uppermost ends in the second electrode group 114 and the third electrode group 116, but arranging these electrodes in parallel with each other along the X axis direction is also preferable.

It is to be noted that the main bodies 62 and 66 of the first and second holding members 52 and 54 having the shapes explained in the first to sixth embodiments can be also allowed. In this case, arranging the respective electrodes of the first electrode group 112 in, e.g., two columns as shown in FIG. 11 can suffice.

### Fifth Embodiment

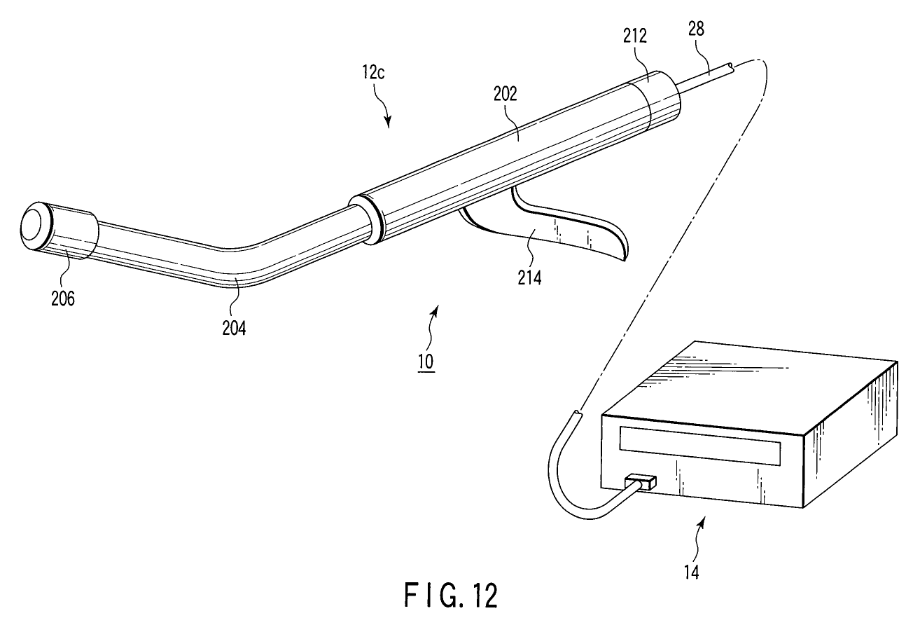

A fifth embodiment will now be explained with reference to FIGS. 12 to 15B.

Here, as an example of an energy treatment device, a circular type bipolar electro-surgical device (a treatment device for curing) 12c will be described which performs a treatment, for example, through an abdominal wall or outside the abdominal wall.

As shown in FIG. 12, the electro-surgical device 12c includes a handle 202, a shaft 204 and an openable/closable holding section 206. The handle 202 is connected with an energy source 14 via a cable 28.

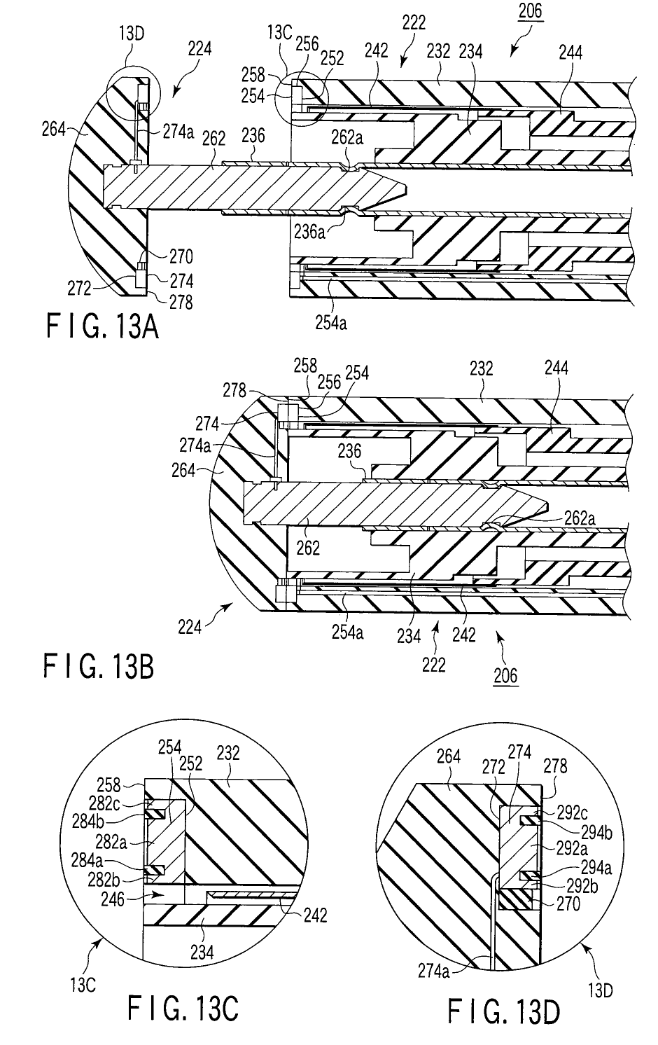

The handle 202 is provided with a holding section opening/closing knob 212 and a cutter driving lever 214. The holding section opening/closing knob 212 is rotatable with respect to the handle 202. When the holding section opening/closing knob 212 is rotated, for example, clockwise with respect to the handle 202, a detachable side holding portion 224 of the holding section 206 described later comes away from a main body side holding portion 222 (see FIG. 13A). When the knob is rotated counterclockwise, the detachable side holding portion 224 comes close to the main body side holding portion 222 (see FIG. 13B).

The shaft 204 is formed into a cylindrical shape. This shaft 204 is appropriately curved in consideration of an insertion property into a living tissue. Needless to say, the shaft 204 may linearly be formed.

A distal end of the shaft 204 is provided with the holding section 206. As shown in FIGS. 13A and 13B, the holding section 206 includes the main body side holding portion (a first holding member) 222 formed at the distal end of the shaft 204, and the detachable side holding portion (a second holding member) 224 detachably attached to the main body side holding portion 222.

The main body side holding portion 222 includes a cylindrical member 232, a frame 234 and an electric conductive pipe 236. The cylindrical member 232 and the frame 234 have an insulating property. The cylindrical member 232 is connected with the distal end of the shaft 204. The frame 234 is fixed to the cylindrical member 232.

A central axis of the frame 234 is opened. The opened central axis of the frame 234 is provided with the electric conductive pipe 236 which is movable in a predetermined region along the central axis of the frame 234. When the holding section opening/closing knob 212 is rotated, as shown in FIGS. 13A and 13B, the electric conductive pipe 236 is movable in a predetermined region owing to, for example, a function of a ball screw (not shown). The electric conductive pipe 236 is provided with a protrusion 236a which protrudes inwards in a diametric direction so that a connecting portion 262a of an electric conductive shaft 262 described later disengageably engages with the protrusion.

As shown in FIGS. 13A and 13B, a space is formed between the cylindrical member 232 and the frame 234. A cylindrical cutter 242 is disposed in the space between the cylindrical member 232 and the frame 234. A proximal end of the cutter 242 is connected with a distal end of a pusher 244 for the cutter disposed in the shaft 204. The cutter 242 is fixed to an outer peripheral surface of the pusher 244 for the cutter. Although not shown, a proximal end of the pusher 244 for the cutter is connected with the cutter driving lever 214 of the handle 202. Therefore, when the cutter driving lever 214 of the handle 202 is operated, the cutter 242 moves via the pusher 244 for the cutter 242.

As shown in FIGS. 13A and 13C, a distal end of the cylindrical member 232 is provided with an annular electrode arrangement portion 252. A first high-frequency electrode 254 is disposed as an output portion or an energy applying portion at the electrode arrangement portion 252. A distal end of a first conducting line 254a is fixed to the first high-frequency electrode ring 254. The first conducting line 254a is connected with the cable 28 via the main body side holding portion 222, the shaft 204 and the handle 202.

As shown in FIGS. 13A, 13C, 14, and 15A, an edge portion 258 is formed on an outer side of the first high-frequency electrode ring 254.

As shown in FIGS. 13C, 14, and 15A, the first high-frequency electrode ring 254 includes a first annular electrode 282a, a second annular electrode 282b, and a third annular electrode 282c. Of these electrodes, the first annular electrode 282a is formed near a central line C between an inner circumference and an outer circumference of the first high-frequency electrode ring 254 (a region near a central axis as a first region). The second annular electrode 282b is formed on an inner side of the first annular electrode 282a (a region away from the central axis as a second region (an inner region of the central axis)). The third annular electrode 282c is formed on an outer side of the first annular electrode 282a (a region away from the central axis as the second region (an outer region of the central axis)). A width of this first annular ring 282a in a radial direction (an R₁ direction) is larger than widths of the second and third annular electrodes 282b and 282c. The widths of the second and third annular electrodes 282b and 282c are substantially equal to each other.

Further, an annular first insulating member 284a is arranged between the first annular electrode 282a and the second annular electrode 282b. An annular second insulating member 284b is arranged between the first annular electrode 282a and the third annular electrode 282c.

The first to third annular electrodes 282a, 282b, and 282c of the first high-frequency electrode ring 254, the first and second insulating members 284a and 284b, and the edge portion 258 of a main body side holding section 222 are a holding surface 222a of the main body side holding section 222 with respect to a living tissue.

On the other hand, as shown in FIGS. 13A and 13B, the detachable side holding portion 224 includes an energization shaft 262 having a connecting portion 262a, and a head portion 264. The energization shaft 262 has a circular cross section, one end formed into a tapered shape, and the other end being fixed to the head portion 264. The connecting portion 262a is formed into a concave groove shape allowing engagement with a protrusion 236a of the energization pipe 236. An outer surface of the energization shaft 262 except the connecting portion 262a is insulated by using, e.g., a coating.

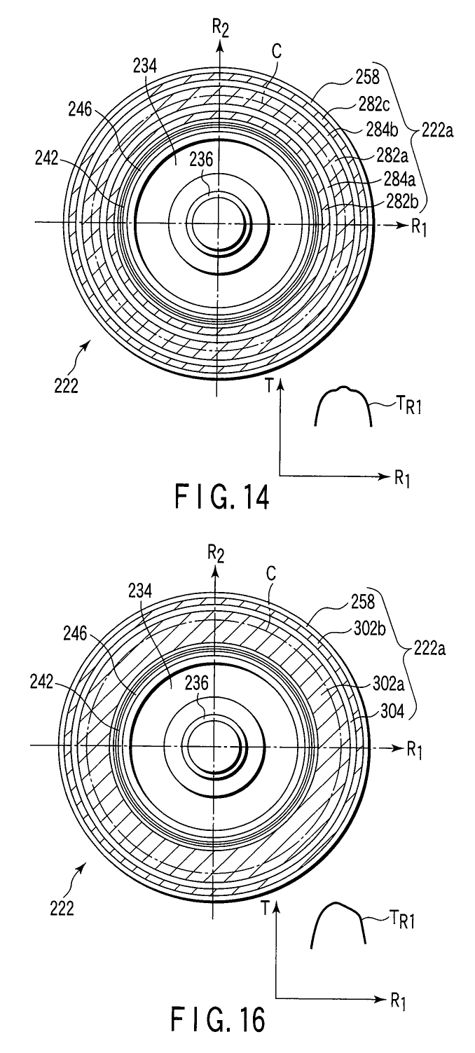

As shown in FIGS. 13A, 13B, 13D, and 14, a cutter receiving portion 270 having an annular shape is provided in the head portion 264. An annular electrode arrangement portion 272 is formed on an outer side of this cutter receiving portion 270. A second high-frequency electrode ring 274 as an output member or an energy applying portion is provided in the electrode arrangement portion 272. One end of a second energization line 274a is fixed to this second high-frequency electrode ring 274. The other end of the second energization line 274a is electrically connected with the energization shaft 262. A contact surface of an edge portion 278 is formed on an outer side of this second high-frequency electrode ring 274.

It is to be noted that the energization pipe 236 is connected with the cable 28 through the shaft 204 and the handle 202. Therefore, when the connecting portion 262a of the energization shaft 262 of the detachable side holding portion 224 is engaged with the protrusion 236a of the energization pipe 236, the second high-frequency electrode ring 274 is electrically connected with the energization pipe 236.

As shown in FIGS. 13D, 14, and 15A, the second high-frequency electrode ring 274 includes a first annular electrode 292a, a second annular electrode 292b, and a third annular electrode 292c. Of these electrodes, the first annular electrode 292a is formed near a central line C between an inner circumference and an outer circumference of the second high-frequency electrode ring 274. The second annular electrode 292b is formed on the inner side of the first annular electrode 292a. The third annular electrode 292c is formed on the outer side of the first annular electrode 292a. A width of the first annular electrode 292a in a radial direction (an R₁ direction) is larger than widths of the second and third annular electrodes 292b and 292c. The widths of the second and third annular electrodes 292b and 292c are substantially equal to each other.

Furthermore, an annular first insulating member 294a is arranged between the first annular electrode 292a and the second annular electrode 292b. An annular second insulating member 294b is arranged between the first annular electrode 292a and the third annular electrode 292c.

A function of a treatment system 10 according to this embodiment will now be explained.

As shown in FIG. 13B, the holding section 206 and the shaft 204 of the electro-surgical device 12c are inserted into, e.g., an abdominal cavity through an abdominal wall in a state where the main body side holding section 222 is closed with respect to the detachable side holding portion 224. The main body side holding portion 222 and the detachable side holding portion 224 of the electro-surgical device 12c is opposed to the living tissue to be treated.

The holding section opening/closing knob 212 of the handle 202 is operated to grasp the living tissue as a treatment target by the main body side holding section 222 and the detachable side holding portion 224. At this time, the holding section opening/closing knob 212 is rotated, e.g., clockwise with respect to the handle 202. Then, as shown in FIG. 13A, the energization pipe 236 is moved to the distal end side with respect to the frame 234 of the shaft 204. Therefore, the space between the main body side holding section 222 and the detachable side holding portion 224 is opened, thereby detaching the detachable side holding portion 224 from the main body side holding section 222.

Moreover, the living tissue as a treatment target is arranged between the first high-frequency electrode ring 254 of the main body side holding section 222 and the second high-frequency electrode ring 274 of the detachable side holding portion 224. The energization shaft 262 of the detachable side holding portion 224 is inserted into the energization pipe 236 of the main body side holding section 222. In this state, the holding section opening/closing knob 212 of the handle 202 is rotated, e.g., counterclockwise. Therefore, the detachable side holding portion 224 is closed with respect to the main body side holding section 222. In this manner, the living tissue as a treatment target is held between the main body side holding section 222 and the detachable side holding portion 224.

In this state, the foot switch or the hand switch is operated, and energy is thereby supplied to the first high-frequency electrode ring 254 and the second high-frequency electrode ring 274 from an energy source 14 via the cable 28. The first to third annular electrodes 282a, 282b, and 282c of the first high-frequency electrode ring 254 apply a high-frequency current to a space between themselves and the first to third annular electrodes 292a, 292b, and 292c of the second high-frequency electrode ring 274 via the living tissue. Therefore, the living tissue between the main body side holding section 222 and the detachable side holding portion 224 is heated.

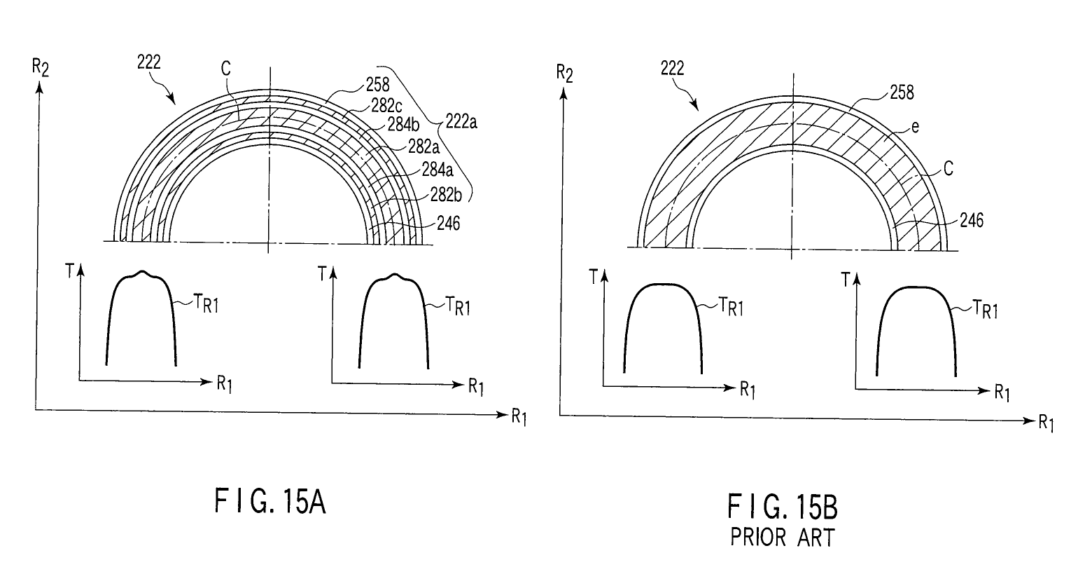

At this time, as shown in FIGS. 14 and 15A, a contact area or a width in a radial direction (an R₁ axis direction in FIGS. 14 and 15A) of the first annular electrode 282a near the central line C with respect to the living tissue is larger than that of the second annular electrode 282b or the third annular electrode 282c away from the central line C. Therefore, energies applied to the living tissue from the second annular electrode 282b and the third annular electrode 282c are smaller than energy supplied to the living tissue from the first annular electrode 282a.

Moreover, the living tissue that is in contact with the second annular electrode 282b or the third annular electrode 282c is away from the central line C and close to the outside of the holding section 206. Therefore, the living tissue is affected by the outside of the holding section 206 having temperature far lower than that of the living tissue present between the main body side holding section 222 and the detachable side holding portion 224. That is, heat of the living tissue grasped by the holding section 206 is taken by a peripheral environment at a position close to the edge portion of the holding section 206.

Therefore, energy density given to the living tissue by the holding section 206 is high at a position close to the central line C, and becomes lower than that at the position close to the central line C as distanced from the central line C. Accordingly, energy distribution of the first annular electrode 282a is higher than those of the second and third annular electrodes 282b and 282c. That is, temperature distribution (energy density) T_{R1} in the R₁ axis direction of the living tissue held by the holding section 206 is high at a position near the central line C, and reduced as distanced from the central line C. Therefore, a temperature gradient in the R₁ axis direction of the living tissue in the holding section 206 is large.

In other words, the living tissue receives large energy at a position near the central line C along the R₁ axis direction of the holding section 206, and receives smaller energy than that at the position near the central line C as away from the central line C. Therefore, when, e.g., welding the living tissue, a treatment of, e.g., denaturing and conjugating the living tissue can be assuredly performed near the central line C of the main body side holding section 222. Contrary, denaturation of a surrounding tissue can be avoided as much as possible.

Additionally, when the cutter driving lever 214 of the handle 202 is operated, a cutter 242 protrudes from a space 246 of the main body side holding section 222 and moves toward a cutter receiving portion 270 of the detachable side holding portion 224. Since the cutter 242 has a blade at a distal end thereof, the treated living tissue is cut into a circular shape.

Meanwhile, one annular electrode e is arranged along the central line C on a holding section 222 on a main side according to the prior art depicted in FIG. 15B. When providing a treatment with respect to a living tissue held by such a holding section 222 on the main side, temperature distribution T_{R1} depicted in FIG. 15B is demonstrated, for example. This temperature distribution T_{R1} is flat at the center in particular.

As explained above, according to this embodiment, the following effects can be obtained.

As depicted in FIG. 15A, the first annular electrode 282a is arranged near the central line C of the first high-frequency electrode ring 254 (see FIG. 13C) of the main body side holding section 222. Further, the contact area of the first annular electrode 282a with respect to the living tissue is set larger than that of the second annular electrode 282b or the third annular electrode 282c. That is, an amount of energy applied to the living tissue from the first annular electrode 282a is set larger than an amount of energy applied to the living tissue from the second annular electrode 282b or the third annular electrode 282c.

Then, a large temperature gradient can be obtained. For example, the temperature distribution T_{R1} in the R₁ axis direction given to the living tissue by the main body side holding section 222 depicted in FIG. 15A is high at a central part (near the central line C) of the main body side holding section 222 and low at a position corresponding to the edge portion as compared with the temperature distribution T_{R1} according to the prior art depicted in FIG. 15B. That is, the temperature gradient of the temperature distribution T_{R1} in the R₁ axis direction given to the living tissue by the main body side holding section 222 depicted in FIG. 15A can be increased beyond a temperature gradient of the temperature distribution T_{R1} according to the prior art shown in FIG. 15B. In particular, the temperature gradient at the central part can be increased. Then, an arrangement of the electrode 282a in the R₁ axis direction of the main body side holding section 222 assuredly enables denaturing and conjugating the living tissue, and an arrangement of the electrodes 282b and 282c can avoid an influence given on a surrounding tissue as much as possible.

It is to be noted that each of the first and the second high-frequency electrode rings 251 and 274 has the annular shape in this embodiment, but various kinds of shapes, e.g., an elliptic shape can be allowed.

### Sixth Embodiment

A sixth embodiment will now be explained with reference to FIG. 16. This embodiment is a modification of the fifth embodiment, and like reference numerals denote members equal to those explained in the fifth embodiment, thereby omitting a detailed explanation.

As shown in FIG. 16, a first high-frequency electrode ring 254 (see FIG. 13C) includes a first annular electrode 302a and a second annular electrode 302b. Of these electrodes, the first annular electrode 302a is arranged on an inner side, and the second annular electrode 302b is arranged on an outer side of the first annular electrode 302a. Further, an annular insulating member 304 is arranged between the first annular electrode 302a and the second annular electrode 302b. It is to be noted that a central line C of the first high-frequency electrode ring 254 is present on the first annular electrode 302a.

At this time, a width of an R₁ axis direction of the first annular electrode 302a is larger than a width in the R₁ axis direction of the second annular electrode 302b. Therefore, energy in the R₁ axis direction applied to a living tissue from the first annular electrode 302a is larger than energy in the R₁ axis direction applied to the living tissue from the second annular electrode 302b.

Furthermore, the living tissue that is in contact with an edge portion on the inner side of the first circular electrode 302a or an edge portion 258 on the outer side of the second circular electrode 302b is away from a central line C and close to the outside of the holding section 206. Therefore, the living tissue is affected by the outside of the holding section 206 having temperature far lower than that of the living tissue present between a main body side holding section 222 and a detachable side holding portion 224. That is, heat of the living tissue grasped by the holding section 206 is taken by peripheral environment at a position close to the edge portion of the holding section 206.

Therefore, energy distribution of the first circular electrode 302a is higher than that of the second circular electrode 302b. That is, temperature distribution (energy density) T_{R1} in an R₁ axis direction of the living tissue held by the holding section 206 is high on the central line C and the inner side of this line, and reduced toward the outer side of the central line C. Therefore, a temperature gradient in the R₁ axis direction of the living tissue in the holding section 206 is large.

In other words, the living tissue receives large energy on the central line C and the inner side of the line along the R₁ axis direction of the holding section 206, and receives smaller energy than that at a position close to the central line C as being away from the central line C. Therefore, when, e.g., welding the living tissue, a treatment of, e.g., denaturing and conjugating the living tissue can be assuredly performed on the central line C and the inner side of this line of the main body side holding section 222. Contrary, denaturation of a surrounding tissue can be avoided as much as possible.

### Seventh Embodiment

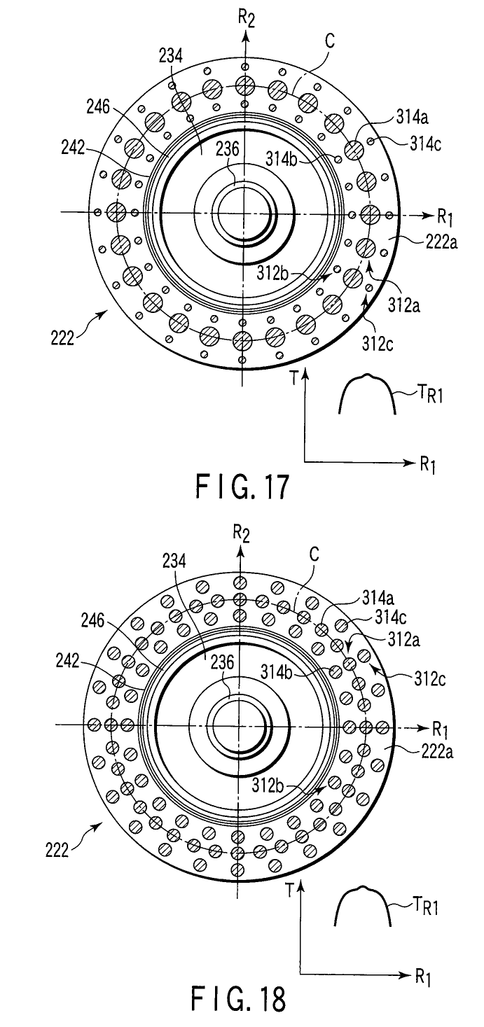

A seventh embodiment will now be explained with reference to FIG. 17. This embodiment is a modification of the fifth embodiment, and like reference numerals denote members equal to those explained in the fifth embodiment, thereby omitting a detailed explanation thereof.

As shown in FIG. 17, a first high-frequency electrode ring 254 (see FIG. 13C) concentrically includes a first annular electrode group 312a, a second annular electrode group 312b, and a third annular electrode group 312c. Of these electrode groups, the first annular electrode group 312a is arranged near a central line C between an inner circumference and an outer circumference of the first high-frequency electrode ring 254. The second annular electrode group 312b is arranged on an inner side of the first annular electrode group 312a. The third annular electrode group 312c is arranged on an outer side of the first annular electrode group 312a.

The first annular electrode group 312a includes a plurality of circular electrodes 314a on the same circumference. The second annular electrode group 312b includes a plurality of circular electrodes 314b on the same circumference. The third annular electrode group 312c includes a plurality of circular electrodes 314c on the same circumference. The electrodes 314a, the electrodes 314b, and the electrodes 314c are aligned in a radial direction, e.g., an R₁ axis direction and an R₂ axis direction. That is, each of the first to third annular electrode groups 312a, 312b, and 312c includes the same number of the electrodes 314a, 314b, or 314c each having the same central angle. Therefore, a length of an arc between centers of the respective electrodes 314a in the first annular electrode group 312a (a distance between centers) is longer than a length of an arc between centers of the respect electrodes 314b in the second annular electrode group 312b. Further, the length of the arc between the centers of the respective electrodes 314a in the first annular electrode group 312a is shorter than a length of an arc between centers of the respective electrodes 314c in the third annular electrode group 312c.

Here, comparing an area or a width in the R₁ axis direction (a diameter) of the electrode 314a in the first annular electrode group 312a with that of the electrode 314b in the second annular electrode group 312, the area or the diameter of the electrode 314a in the first annular electrode group 312a is larger. The area or the diameter of the electrode 314b in the second annular electrode group 312b is substantially equal to that of the electrode 314c in the third annular electrode group 312c. Therefore, energy applied to a living tissue from each electrode 314a in the first annular electrode group 312a is larger than energies applied to the living tissue from each electrode 314b and each electrode 314c in the second annular electrode group 312b and the third annular electrode group 312c.

Moreover, the living tissue that is in contact with the second annular electrode group 312b or the third annular electrode group 312c is away from a central line C and close to the outside of a holding section 206. Therefore, the living tissue is affected by the outside of the holding section 206 having temperature far lower than that of the living tissue present between a main body side holding section 222 and a detachable side holding portion 224. That is, heat of the living tissue held by the holding section 206 is taken by a peripheral environment at a position close to an edge portion of the holding section 206.

Therefore, energy distribution of the first annular electrode group 312a is higher than energy distributions of the second and third annular electrode groups 312b and 312c. That is, temperature distribution (energy density) T_{R1} in the R₁ axis direction of the living tissue grasped by the holding section 206 is high at a position near the central line C, and reduced as away from the central line C. Therefore, a temperature gradient in the R₁ axis direction of the living tissue in the holding section 206 is large.

In other words, the living tissue receives large energy at a position near the central line C along the R₁ axis direction of the holding section 206, and receives smaller energy than that at a position near the central line C as away from the central line C. Therefore, when, e.g., welding the living tissue, a treatment of, e.g., denaturing and conjugating the living tissue can be assuredly performed near the central line C of the main body side holding section 222. Contrary, denaturation of a surrounding tissue can be avoided as much as possible.

It is to be noted that the example where the first annular electrode group 312a includes the plurality of electrodes 314a has been explained in this embodiment, but a structure where the first annular electrode group 312a is formed into a continuous annular shape like the first annular electrode 282a (see FIG. 13C) explained in the fifth embodiment is also preferable.

### Eighth Embodiment

An eighth embodiment will now be explained with reference to FIG. 18. This embodiment is a modification of the fifth embodiment, and like reference numerals denote members equal to those explained in the fifth embodiment, thereby omitting a detailed explanation.

As shown in FIG. 18, a first high-frequency electrode ring 254 (see FIG. 13C) concentrically includes a first annular electrode group 312a, a second annular electrode group 312b, and a third annular electrode group 312c. Of these electrode groups, the first annular electrode group 312a is arranged near a central line C between an inner circumference and an outer circumference of the first high-frequency electrode ring 254. The second annular electrode group 312b is arranged on an inner side of the first annular electrode group 312a. The third annular electrode group 312c is arranged on an outer side of the first annular electrode group 312a.

The first annular electrode group 312a includes a plurality of circular electrodes 314a on the same circumference. The second annular electrode group 312b includes a plurality of circular electrodes 314b on the same circumference. The third annular electrode group 312c includes a plurality of circular electrodes 314c on the same circumference. The electrodes 314b in the second annular electrode group 312b and the electrodes 314c in the third annular electrode group 312c are aligned in a radial direction, e.g., an R₁ axis direction and an R₂ axis direction. Additionally, each of the second and third annular electrode groups 312b and 312c includes the same number of the electrodes 314b or 314c. The number of the electrodes 314a in the first annular electrode group 312a is increased to be approximately 1.5-fold of the number of the electrodes 314b or 314c in the second or the third annular electrode group 312b or 312c.

It is to be noted that the electrodes 314a, 314b, and 314c in the first to third annular electrode groups 312a, 312b, and 312c have the same area.

Therefore, a length of an arc between centers of the respective electrodes 314a in the first annular electrode group 312a (a distance between centers) is shorter than a length of an arc between centers of the respective electrodes 314b in the second annular electrode group 312b. Further, the length of the arc between the centers of the respective electrodes 314a in the first annular electrode group 312a is shorter than a length of an arc between centers of the respective electrodes 314c in the third annular electrode group 312c. Therefore, density of the first annular electrode group 312a is higher than those of the second and third annular electrode groups 312b and 312c.

Furthermore, a living tissue that is in contact with the second annular electrode group 312b or the third annular electrode group 312c is away from the central line C and close to the outside of a holding section 206. Therefore, the living tissue is affected by the outside of the holding section 206 having temperature far lower than that of the living tissue present between the main body side holding section 222 and the detachable side holding portion 224. That is, heat of the living tissue grasped by the holding section 206 is taken by a peripheral environment at a position near an edge portion of the holding section 206.

Therefore, energy distribution of the first annular electrode group 312a is higher than energy distributions of the second and third annular electrode groups 312b and 312c. That is, temperature distribution (energy density) T_{R1} in the R₁ axis direction of the living tissue held by the holding section 206 is high at a position near the central line C, and reduced as distanced from the central line C. Therefore, a temperature gradient along the R₁ axis direction of the living tissue in the holding section 206 is large.

In other words, the living tissue receives large energy at a position near the central line C and receives smaller energy than that at the position near the central line C as distanced from the central line C in the R₁ axis direction of the holding section 206. Therefore, when, e.g., welding the living tissue, a treatment of, e.g., denaturing and conjugating the living tissue can be assuredly performed near the central line C of the main body side holding section 222. Contrary, denaturation of a surrounding tissue can be avoided as much as possible.

### Ninth Embodiment

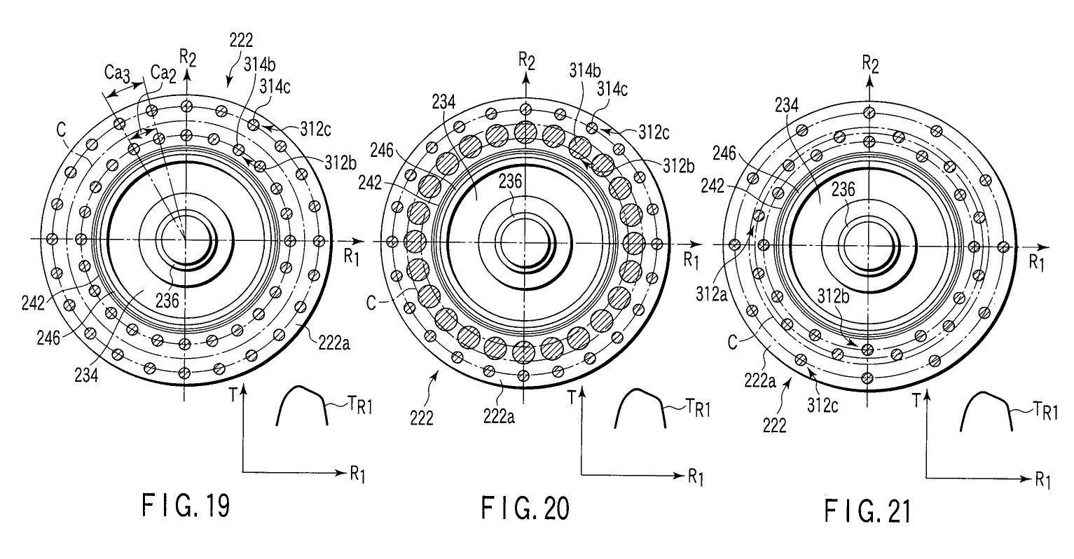

A ninth embodiment will now be explained with reference to FIG. 19. This embodiment is a modification of the fifth to eighth embodiments, and like reference numerals denote members equal to those explained in the fifth to eighth embodiments, thereby omitting a detailed explanation.

As shown in FIG. 19, in this embodiment, the first annular electrode group 312a in the eighth embodiment is removed. A length (a distance between centers) Cₐ₂ of an arc between electrodes 314b adjacent each other in a second annular electrode group (an inner circumferential region of a main body side holding section 222) 312b is shorter than a length (a distance between centers) Cₐ₃ of an arc between electrodes 314c adjacent to each other in a third annular electrode group (an outer circumferential region of the main body side holding section 222) 312c. At this time, each electrode 314b in the second annular electrode group 312b has the same diameter and the same area as those of each electrode 314c in the third annular electrode group 312c. Therefore, energies in an R₁ axis direction applied to a living tissue from the second annular electrode group 312b and the third annular electrode group 312c are substantially equal to each other.

However, as explained above, the distance Cₐ₃ between the electrodes 314c adjacent to each other in the third annular electrode group 312c is longer than the distance Cₐ₂ between the electrodes 314b adjacent to each other in the second annular electrode group 312b. Therefore, density of the third annular electrode group 312c is lower than that of the second annular electrode group 312b. Then, energy applied to the living tissue from each electrode 314b in the second annular electrode group 312b is larger than energy applied to the living tissue from each electrode 314c in the third annular electrode group 312c.

Further, the living tissue that is in contact with an outer edge portion of the third annular electrode group is away from a central line C and close to the outside of a holding section 206. Therefore, the living tissue is affected by the outside of the holding section 206 having temperature far lower than that of the living tissue present between the main body side holding section 222 and a detachable side holding portion 224. That is, heat of the living tissue held by the holding section 206 is taken by a surrounding environment at a position close to an edge portion of the holding section 206.

Therefore, energy distribution of the second annular electrode group 312b is higher than energy distribution of the third annular electrode group 312c. That is, temperature distribution (energy density) T_{R1} in an R₁ axis direction of the living tissue grasped by the holding section 206 is high on the central line C and an inner side of this line, and reduced as being away from the central line C. Therefore, a temperature gradient in the R₁ axis direction of the living tissue in the holding section 206 is large.

In other words, the living tissue receives large energy on the central line C and the inner side of this line along the R₁ axis direction of the holding section 206, and receives smaller energy than that at a position near the central line C as being away from the central line C. Therefore, when, e.g., welding the living tissue, a treatment of, e.g., denaturing and conjugating the living tissue can be assuredly performed on the central line C and the inner side of this line of the main body side holding section 222. Contrary, denaturation of a surrounding tissue can be avoided as much as possible.

### Tenth Embodiment

A tenth embodiment will now be explained with reference to FIG. 20. This embodiment is a modification of the sixth and the ninth embodiments, and like reference numerals denote members equal to those explained in the sixth and the ninth embodiments, thereby omitting a detailed explanation.

As shown in FIG. 20, an area of each electrode 314b in a second annular electrode group 312b is different from an area of each electrode 314c in a third annular electrode group 312c as different from the ninth embodiment. Each electrode in the second annular electrode group 312b in this example has a diameter and an area larger than those of each electrode 314b in the second annular electrode group 312b explained in the ninth embodiment.

Other structures, functions, and effects are the same as those in the sixth embodiments, thereby omitting an explanation thereof.

### Eleventh Embodiment

An Eleventh embodiment will now be explained with reference to FIG. 21. This embodiment is a modification of the sixth to tenth embodiments, and like reference numerals denote members equal to those explained in the sixth to tenth embodiments, thereby omitting a detailed explanation.

As shown in FIG. 21, the number of respective electrodes 314b in a second annular electrode group 312b is equal to the number of respective electrodes 314c in a third annular electrode group 312c. However, the number of the respective electrodes 314b and 314c in the second and third annular electrode groups 312b and 312c is reduced to approximately ½ to ⅓ of that explained in the eighth embodiment.

Furthermore, each electrode 314a in a first annular electrode group 312a is arranged on an inner side apart from a central line C. In this embodiment, each electrode 314a in the first annular electrode group 312a is inscribed with respect to the central line C. That is, each electrode 314a in the first annular electrode group 312a is arranged at a position that is slightly close to the inner side apart from the central line C, and approximates each electrode 314b in the second annular electrode group 312b. Moreover, each electrode 314a in the first annular electrode group 312a is arranged between the respective electrodes 314b and 314c in the second and third annular electrode groups 312b and 312c.

Therefore, on a holding surface 222a of a main body side holding section 222, the first and the second annular electrode groups 312a and 312b on the inner side apart from the central line C have high densities, and the third annular electrode group 312c on the outer side has low density.

Additionally, a living tissue that is in contact with an outer edge portion of the third annular electrode group 312c is away from the central line C and close to the outside of a holding section 206. Therefore, the living tissue is affected by the outside of the holding section 206 having temperature far lower than that of the living tissue present between the main body side holding section 222 and a detachable side holding portion 224. That is, heat of the living tissue held by the holding section 206 is taken by a peripheral environment at a position close to an edge portion of the holding section 206.

Therefore, energy distributions of the first and the second annular electrode groups 312a and 312b are higher than energy distribution of the third annular electrode group 312c. That is, temperature distribution (energy density) T_{R1} in an R₁ axis direction of the living tissue held by the holding section 206 is high on the central line C and the inner side of this line, and reduced as being away from the central line C. Therefore, a temperature gradient in the R₁ axis direction of the living tissue in the holding section 206 is large.

In other words, the living tissue receives large energy on the central line C and the inner side of this line along the R₁ axis direction of the holding section 206, and receives smaller energy than that at a position near the central line C as being away from the central line C. Therefore, when, e.g., welding the living tissue, a treatment of, e.g., denaturing and conjugating the living tissue can be assuredly performed on the central line C and the inner side of this line of the main body side holding section 222. Contrary, denaturation of a surrounding tissue can be avoided as much as possible.

It is to be noted that the example where each of the electrodes 314a, 314b, and 314c has the circular shape has been explained in the seventh to eleventh embodiments, various kinds of shapes, e.g., an elliptic shape or a rhombic shape can be allowed.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A treatment system that applies energy to a living tissue, comprising:
first and second holding members each having a holding surface to hold the living tissue;
an operating section that operates a relative movement of at least one of the first and second holding members with respect to the other;
an energy source that supplies energy to at least one of the first and second holding members; and
a plurality of energy applying portions that apply energy supplied from the energy source, the plurality of energy applying portions being provided on the holding surface of at least one of the first and second holding members and controlling density of energy applied to the living tissue held by the first and second holding members.

2. The system according to claim 1,
wherein each of the first and second holding members includes a proximal end, a distal end, and a central axis in a longitudinal direction,
the plurality of energy applying portions include a first region arranged near the central axis of the holding surface of at least one of the first and second holding members, and a second region arranged at a position away from the central axis, and
energy density of the energy applying portions in the first region is larger than energy density of the energy applying portions in the second region.

3. The system according to claim 2,
wherein the plurality of energy applying portions are arranged like spots in each of the first region and the second region, and
the number of the energy applying portions arranged in the first region is larger than the number of the energy applying portions arranged in the second region.

4. The system according to claim 2,
wherein the plurality of energy applying portions are arranged like spots in each of the first and the second regions, and
a gap between the energy applying portions arranged in the first region is smaller than a gap between the energy applying portions arranged in the second region.

5. The system according to claim 2,
wherein the plurality of energy applying portions are arranged like spots in each of the first and second regions, and
an area of each energy applying portion arranged in the first region is equal to or larger than an area of each energy applying portion arranged in the second region.

6. The system according to claim 1,
wherein each of the first and second holding members has an annular shape having an inner circumference, an outer circumference, and a central line between the inner circumference and the outer circumference,
the plurality of energy applying portions include a first region arranged near the central line and a second region away from the central line, and
energy density of the energy applying portions in the first region is larger than energy density of the energy applying portions in the second region.

7. The system according to claim 6,
wherein the second region includes an inner region on an inner side of the first region and an outer region on an outer side of the first region, and
a width in a radial direction of each energy applying portions in the first region is larger than a width in the radial direction of each energy applying portion in each of the inner region and the outer region.

8. The system according to claim 6,
wherein the first region is arranged on the inner circumference including the central line of at least one of the first and second holding members,
the second region is arranged on the outer side of the first region, and
energy density of the energy applying portions in the first region is larger than energy density of the energy applying portions in the second region.

9. The system according to claim 6,
wherein the second region includes an inner region on an inner side of the first region and an outer region on an outer side of the first region,
the plurality of energy applying portions arranged in the first region, the inner region, and the outer region are respectively concentrically arranged, and
the number of the energy applying portions arranged in the first region is larger than the number of the energy applying portions arranged in each of the inner region and the outer region.

10. The system according to claim 6,
wherein the second region includes an inner region on an inner side of the first region and an outer region on an outer side of the first region,
the plurality of energy applying portions arranged in the first region, the inner region, and the outer region are respectively concentrically arranged, and
an area of each energy applying portions arranged in the first region is equal to or larger than an area of each energy applying portions arranged in the inner region and the outer region.

11. The system according to claim 6,
wherein the second region includes an inner region on an inner side of the first region and an outer region on an outer side of the first region,
the plurality of energy applying portions arranged in the first region, the inner region, and the outer region are respectively concentrically arranged, and
a gap between the energy applying portions arranged in the first region is smaller than a gap between the energy applying portions arranged in each of the inner region and the outer region.

12. The system according to claim 1,
wherein each of the first and second holding members has an annular shape having an inner circumference, an outer circumference, and a central line between the inner circumference and the outer circumference,
the energy applying portions include an inner circumferential region arranged on the inner circumference side of at least one of the first and second holding members including a position near the central line, and an outer circumferential region arranged on an outer side of the inner circumferential region, and
energy density of the energy applying portions in the inner circumferential region is larger than energy density of the energy applying portions in the outer circumferential region.

13. A treatment device that applies energy to a living tissue, comprising:
a holding section that holds the living tissue, the holding section including:
first and second holding members that are relatively movable with respect to each other; and
a plurality of energy applying portions that are provided on at least one of the first and second holding members and connected with an energy source, the energy applying portions being provided on at least one of the first and second holding members and controlling density of energy applied to the living tissue when applying the energy to the living tissue held by the first and second holding members.

14. The device according to claim 13,
wherein each of the first and second holding members includes a proximal end, a distal end, a central axis in a longitudinal direction, and a holding surface arranged at a position close to the other holding member,
the plurality of energy applying portions include a first region arranged near the central axis on the holding surface of at least one of the first and second holding members, and second and third regions arranged at positions away from the central axis, and
energy density of the energy applying portions in the first region is larger than energy densities of the energy applying portions in the second and third regions.

15. The device according to claim 14,
wherein the plurality of energy applying portions are arranged like spots in each of the first to third regions, and
the number of the energy applying portions arranged in the first region is larger than the number of the energy applying portions arranged in each of the second and third regions.

16. The device according to claim 14,
wherein the plurality of energy applying portions are arranged like spots in each of the first to third regions, and
a gap between the energy applying portions arranged in the first region is smaller than a gap between the energy applying portions arranged in each of the second and third regions.

17. The device according to claim 14,
wherein the plurality of energy applying portions are arranged like spots in each of the first to third regions, and
an area of each energy applying portion arranged in the first region is equal to or larger than an area of each energy applying portions arranged in each of the second and third regions.

18. The device according to claim 13,
wherein each of the first and second holding members has an annular shape having an inner circumference, an outer circumference, and a central line between the inner circumference and the outer circumference,
the energy applying portions include a first region arranged near the central line, and a second region away from the central line, and
energy density of the energy applying portions in the first region is larger than energy density of the energy applying portions in the second region.

19. The device according to claim 18,
wherein the second region includes an inner region on an inner side of the first region, and an outer region on an outer side of the first region, and
a width in a radial direction of each energy applying portions in the first region is larger than a width in the radial direction of each energy applying portions in the inner region and the outer region.

20. The device according to claim 18,
wherein the first region is arranged on the inner circumference including the central line of at least one of the first and second holding members,
the second region is arranged on the outer side of the first region, and
energy density of the energy applying portions in the first region is larger than energy density of the energy applying portions in the second region.

21. The device according to claim 18,
wherein the second region includes an inner region on an inner side of the first region, and an outer region on an outer side of the first region,
the plurality of energy applying portions arranged in the first region, the inner region, and the outer region are respectively concentrically arranged, and
the number of the energy applying portions arranged in the first region is larger than the number of the energy applying portions arranged in each of the inner region and the outer region.

22. The device according to claim 18,
wherein the second region includes an inner region on an inner side of the first region, and an outer region on an outer side of the first region,
the plurality of energy applying portions are concentrically arranged in each of the first region, the inner region, and the outer region, and
an area of each energy applying portions arranged in the first region is equal to or larger than an area of each energy applying portion arranged in each of the inner region and the outer region.

23. The device according to claim 18,
wherein the second region includes an inner region on an inner side of the first region, and an outer region on an outer side of the first region,
the plurality of energy applying portions arranged in each of the first region, the inner region, and the outer region are concentrically arranged, and
a gap between the energy applying portions arranged in the first region is smaller than a gap between the energy applying portions arranged in each of the inner region and the outer region.

24. The device according to claim 13,
wherein each of the first and second holding members has an annular shape having an inner circumference, an outer circumference, and a central line between the inner circumference and the outer circumference,
the energy applying portions include an inner circumferential region arranged on an inner circumferential side of at least one of the first and second holding members including a position near the central line, and an outer circumferential region arranged on an outer side of the inner circumferential region, and
energy density of the energy applying portions in the inner circumferential region is larger than energy density of the energy applying portions in the outer circumferential region.

25. A treatment method for a living tissue using an energy, comprising:
holding the living tissue;
applying energy to the living tissue to denature the living tissue; and
increasing energy density at a desired position where the held living tissues denatures by the energy applied to the living tissue.
